SEQUENCE LISTING (79 sequences)

SEQ ID NO: 1 - protein, 463 aa, Thermococcus gammatolerans
MRRYTRVLILLMALFLLAGLYYPSASAAKYSELEQGGVIMQAFYWDVPAGGIWWDTIRQKIPEWYDAGISAIWIPPASKGMGGAYSMGYDPYDYFDLGEFYQKGTVETRFGSKEELVNMISTAHRYGIKVIADIVINHRAGGDLEWNPYVGDYTWTDFSQVASGKYKAHYMDFHPNNYSTSDEGTFGGFPDIDHLVPFNKYWLWASDESYAAYLRSIGVDAWRFDYVKGYGAWVVKDWLSWWGGWAVGEYWDTDVNALLNWAYDSGAKVFDFPLYYKMDEAFDNKNIPALVYAIQNGGTVVSRDPFKAVTFVANHDTNIIWNKYPAYAFILTYEGQPVIFYRDYEEWLNKDKLNNLIWIHEHLAGGSTKILYYDDDELIFMREGYGDRPGLITYINLGSGWAERWVNVGSKFAGYTIHEYTGNLGGWVDRYVYYNGWVKLTAPPHDPANGYYGYSVWSYAGVG

SEQ ID NO: 2 - protein, 457 aa, Thermococcus thioreducens
MARKVTVALLVLLVVLSLSAVPAKAETLENGGVIMQAFYWDVPMGGIWWDTIAQKIPDWASAGISAIWIPPASKGMSGGYSMGYDPYDYFDLGEYYQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASNESYAAYLRSIGIDAWRFDYVKGYAPWVVKDWLNWWGGWAVGEYWDTNVDALLNWAYASGAKVFDFPLYYKMDEAFDNNNIPALVDALRYGQTVVSRDPFKAVTFVANHDTDIIWNKYPAYAFILTYEGQPMIFYRDYEEWLNKDRLKNLIWIHDHLAGGSTDIVYYDSDELIFVRNGYGSKPGLITYINLGSSKAGRWVYVPKFAGSCIHEYTGNLGGWVDKWVDSSGWVYLEAPAHDPANGQYGYSVWSYCGVG

SEQ ID NO: 3 - protein, 461 aa, thermococcus eurythermalis
MKPAKLLVFVLVVSILAGLYAQPAGAAKYLELEEGGVIMQAFYWDVPSGGIWWDTIRQKIPEWYDAGISAIWIPPASKGMGGAYSMGYDPYDFFDLGEYDQKGTVETRFGSKQELVNMINTAHAYGIKVIADIVINHRAGGDLEWNPFVNDYTWTDFSKVASGKYTANYLDFHPNEVKCCDEGTFGGFPDIAHEKSWDQYWLWASNESYAAYLRSIGVDAWRFDYVKGYGAWVVKDWLDWWGGWAVGEYWDTNVDALLNWAYSSDAKVFDFPLYYKMDAAFDNKNIPALVEALKNGGTVVSRDPFKAVTFVANHDTDIIWNKYPAYAFILTYEGQPTIFYRDYEEWLNKDRLKNLIWIHDHLAGGSTDIVYYDNDELIFVRNGYGDKPGLITYINLGSSKAGRWVYVPKFAGACIHEYTGNLGGWVDKWVDSSGWVYLEAPAHDPANGYYGYSVWSYCGVG

SEQ ID NO: 4 - protein, 457 aa, Thermococcus hydrothermalis
MARKVLVALLVFLVVLSVSAVPAKAETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 5 - protein, 460 aa, Pyrococcus furiosus
MNIKKLTPLLTLLLFFIVLASPVSAAKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 6 - DNA, 807 bp, Artificial Sequence
  SED1 tether
  CDS (1)..(807)
aaggacaatagctcgacgattgaaggtagatacccatacgacgttccagactacgctctgcaggctagtggtggtggtggttctggtggtggtggttctggtggtggtggttctgctagcgctcttccaactaacggtacttctactgaagctccaactgatactactactgaagctccaaccaccggtcttccaaccaacggtaccacttcagctttcccaccaactacatctttgccaccaagcaacactaccaccactcctccttacaacccatctactgactacaccactgactacactgtagtcactgaatatactacttactgtccagaaccaaccactttcaccacaaacggtaagacttacaccgtcactgaaccaaccacattgactatcactgactgtccatgcaccattgaaaagccaacaaccacatcaaccaccgaatacactgtagtcactgagtacactacttactgtccagaaccaaccactttcaccacaaacggtaagacttacaccgtcactgaaccaaccactttgactatcactgactgtccatgtactattgaaaagagcgaagcccctgagtcttctgtcccagttaccgaatctaagggcactaccaccaaagaaacaggtgttactaccaaacaaaccacagccaacccaagtctaaccgtctccacagtcgtcccagtttcatcctctgcttcttctcattccgttgtcatcaacagtaacggtgctaacgtcgtcgttccaggtgctttaggtttggctggtgttgctatgttattctaa

SEQ ID NO: 7 - protein, 268 aa, Artificial Sequence
  Synthetic Construct
KDNSSTIEGRYPYDVPDYALQASGGGGSGGGGSGGGGSASALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLF

SEQ ID NO: 8 - DNA, 390 bp, Artificial Sequence
  SPI1 tether
  CDS (1)..(390)
ttggtatctaattctagttcctctgtaatcgtggtaccatcaagcgatgctactattgccggtaacgatacagccacgccagcaccagagccatcatccgccgctccaatattctacaactcgactgctactgcaacacagtacgaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 9 - protein, 129 aa, Artificial Sequence
  Synthetic Construct
LVSNSSSSVIVVPSSDATIAGNDTATPAPEPSSAAPIFYNSTATATQYEVVSEFTTYCPEPTTFVTNGATFTVTAPTTLTITNCPCTIEKPTSETSVSSTHDVETNSNAANARAIPGALGLAGAVMMLL

SEQ ID NO: 10 - DNA, 339 bp, Artificial Sequence
  CCW12 tether
  CDS (1)..(339)
gttaccactgctactgtcagccaagaatctaccactttggtcaccatcacttcttgtgaagaccacgtctgttctgaaactgtctccccagctttggtttccaccgctaccgtcaccgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 11 - protein, 112 aa, Artificial Sequence
  Synthetic Construct
VTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 12 - DNA, 450 bp, Artificial Sequence
  CWP2 tether
  CDS (1)..(450)
aaggacaatagctcgacgattgaaggtagatacccatacgacgttccagactacgctctgcaggctagtggtggtggtggttctggtggtggtggttctggtggtggtggttctgctagcggatccggtggcggtggatctggaggaggcggttcttggtctcacccacaatttgaaaagggtggagaaaacttgtactttcaaggcggtggtggaggttctggcggaggtggctccggctcagctatctctcaaatcaccgacggtcaaatccaagccactaccacagctaccactgaagctacaactaccgctgctccttcatctactgttgaaactgtttctccatcttccaccgaaaccatctctcaacaaaccgaaaacggtgctgctaaggctgctgttggtatgggtgctggtgctttggctgctgctgctatgttgttgtaa

SEQ ID NO: 13 - protein, 149 aa, Artificial Sequence
  Synthetic Construct
KDNSSTIEGRYPYDVPDYALQASGGGGSGGGGSGGGGSASGSGGGGSGGGGSWSHPQFEKGGENLYFQGGGGGSGGGGSGSAISQITDGQIQATTTATTEATTTAAPSSTVETVSPSSTETISQQTENGAAKAAVGMGAGALAAAAMLL

SEQ ID NO: 14 - DNA, 759 bp, Artificial Sequence
  TIR1 tether
  CDS (1)..(759)
aaggacaatagctcgacgattgaaggtagatacccatacgacgttccagactacgctctgcaggctagtggtggtggtggttctggtggtggtggttctggtggtggtggttctgctagcagcttagcttctgattcttcctctggattttccttaagcagtatgccagctggtgttttggatatcggtatggctttagcttccgccactgacgactcctacactactttgtactctgaggttgactttgctggtgttagcaagatgttgaccatggttccatggtactcctctagattggaaccagctttgaagtctttgaatggtgatgcttcttcttctgctgccccaagctcttctgctgctccaacttcttctgctgccccaagctcatctgctgccccaacttcttctgctgcctcaagctcttctgaagctaagtcttcttctgctgccccaagctcttctgaagctaagtcttcttctgctgccccaagctcttctgaagctaagtcttcttctgctgccccaagctcttctgaagctaagtcttcttctgctgctccaagctccactgaagctaagataacttctgctgctccaagctccactggtgccaagacctctgccatctctcaaattaccgatggtcaaatccaagctaccaaggctgtttctgagcaaactgaaaacggtgctgctaaggcctttgttggtatgggtgctggtgttgtcgcagctgccgctatgttgttataa

SEQ ID NO: 15 - protein, 252 aa, Artificial Sequence
  Synthetic Construct
KDNSSTIEGRYPYDVPDYALQASGGGGSGGGGSGGGGSASSLASDSSSGFSLSSMPAGVLDIGMALASATDDSYTTLYSEVDFAGVSKMLTMVPWYSSRLEPALKSLNGDASSSAAPSSSAAPTSSAAPSSSAAPTSSAASSSSEAKSSSAAPSSSEAKSSSAAPSSSEAKSSSAAPSSSEAKSSSAAPSSTEAKITSAAPSSTGAKTSAISQITDGQIQATKAVSEQTENGAAKAFVGMGAGVVAAAAMLL

SEQ ID NO: 16 - DNA, 1398 bp, Artificial Sequence
  PST1 tether
  CDS (1)..(1398)
aaggacaatagctcgacgattgaaggtagatacccatacgacgttccagactacgctctgcaggctagtggtggtggtggttctggtggtggtggttctggtggtggtggttctgctagcgctacttcctcttcttccagcataccctcttcctgtaccataagctcacatgccacggccacagctcagagtgacttagataaatatagccgctgtgatacgttagtcgggaacttaactattggtggtggtttgaagactggtgctttggctaatgttaaagaaatcaacgggtctctaactatatttaacgctacaaatctaacctcattcgctgctgattccttggagtccatcacagattctttgaacctacagagtttgacaatcttgacttctgcttcatttgggtctttacagagcgttgatagtataaaactgattactctacccgccatctccagttttacttcaaatatcaaatctgctaacaacatttatatttccgacacttcgttacaatctgtcgatggattctcagccttgaaaaaagttaacgtgttcaacgtcaataacaataagaaattaacctcgatcaaatctccagttgaaacagtcagcgattctttacaattttcgttcaacggtaaccagactaaaatcaccttcgatgacttggtttgggcaaacaatatcagtttgaccgatgtccactctgtttccttcgctaacttgcaaaagattaactcttcattgggtttcatcaacaactccatctcaagtttgaatttcactaagctaaacaccattggccaaaccttcagtatcgtttccaatgactacttgaagaacttgtcgttctctaatttgtcaaccataggtggtgctcttgtcgttgctaacaacactggtttacaaaaaattggtggtctcgacaacctaacaaccattggcggtactttggaagttgttggtaacttcacctccttgaacctagactctttgaagtctgtcaagggtggcgcagatgtcgaatcaaagtcaagcaatttctcctgtaatgctttgaaagctttgcaaaagaaagggggtatcaagggtgaatcttttgtctgcaaaaatggtgcatcatccacatctgttaaactatcgtccacttccaaatctcaatcaagccaaactactgccaaggtttccaagtcatcttctaaggccgaggaaaagaagttcacttctggcgatatcaaggctgctgcttctgcctctagtgtttctagttctggcgcttccagctctagctctaagagttccaaaggcaatgccgctatcatggcaccaattggccaaacaacccctttggtcggtcttttgacggcaatcatcatgtctataatgtaa

SEQ ID NO: 17 - protein, 465 aa, Artificial Sequence
  Synthetic Construct
KDNSSTIEGRYPYDVPDYALQASGGGGSGGGGSGGGGSASATSSSSSIPSSCTISSHATATAQSDLDKYSRCDTLVGNLTIGGGLKTGALANVKEINGSLTIFNATNLTSFAADSLESITDSLNLQSLTILTSASFGSLQSVDSIKLITLPAISSFTSNIKSANNIYISDTSLQSVDGFSALKKVNVFNVNNNKKLTSIKSPVETVSDSLQFSFNGNQTKITFDDLVWANNISLTDVHSVSFANLQKINSSLGFINNSISSLNFTKLNTIGQTFSIVSNDYLKNLSFSNLSTIGGALVVANNTGLQKIGGLDNLTTIGGTLEVVGNFTSLNLDSLKSVKGGADVESKSSNFSCNALKALQKKGGIKGESFVCKNGASSTSVKLSSTSKSQSSQTTAKVSKSSSKAEEKKFTSGDIKAAASASSVSSSGASSSSSKSSKGNAAIMAPIGQTTPLVGLLTAIIMSIM

SEQ ID NO: 18 - DNA, 330 bp, Artificial Sequence
  Aga1/2 tether
  CDS (1)..(330)
aaggacaatagctcgacgattgaaggtagatacccatacgacgttccagactacgctctgcaggctagtggtggtggtggttctggtggtggtggttctggtggtggtggttctgctagccaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttaa

SEQ ID NO: 19 - protein, 109 aa, Artificial Sequence
  Synthetic Construct
KDNSSTIEGRYPYDVPDYALQASGGGGSGGGGSGGGGSASQELTTICEQIPSPTLESTPYSLSTTTILANGKAMQGVFEYYKSVTFVSNCGSHPSTTSKGSPINTQYVF

SEQ ID NO: 20 - DNA, 381 bp, Artificial Sequence
  Aga1/2 tether
  CDS (1)..(381)
atgcagttacttcgctgtttttcaatattttctgttattgcttcagttttagcacaggagctgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcgattctcacccctcaacaactagcaaagacagccccataaacacacagtatgtttttaaggacaatagctcgacgattgaaggtagatacccatacgacgttccagactacgctctgcaggctagtggtggtggtggttctggtggtggtggttctggtggtggtggttctgctagc

SEQ ID NO: 21 - protein, 127 aa, Artificial Sequence
  Synthetic Construct
MQLLRCFSIFSVIASVLAQELTTICEQIPSPTLESTPYSLSTTTILANGKAMQGVFEYYKSVTFVSNCDSHPSTTSKDSPINTQYVFKDNSSTIEGRYPYDVPDYALQASGGGGSGGGGSGGGGSAS

SEQ ID NO: 22 - protein, 15 aa, Artificial Sequence
  Linker
GGGGSGGGGSGGGGS

SEQ ID NO: 23 - protein, 8 aa, Artificial Sequence
  Linker
GGGGGGGG

SEQ ID NO: 24 - protein, 40 aa, Artificial Sequence
  Linker
GGGGSGGGGSGGGGSGGGGSGGGGSGGGGSGGGGSGGGGS

SEQ ID NO: 25 - protein, 12 aa, Artificial Sequence
  Linker
GSAGSAAGSGEF

SEQ ID NO: 26 - protein, 12 aa, Artificial Sequence
  Linker
EAAKEAAKEAAK

SEQ ID NO: 27 - protein, 20 aa, Artificial Sequence
  Linker
APAPAPAPAPAPAPAPAPAP

SEQ ID NO: 28 - protein, 46 aa, Artificial Sequence
  Linker
AEAAAKEAAAKEAAAKEAAAKALEAEAAAKEAAAKEAAAKEAAAKA

SEQ ID NO: 29 - DNA, 66 bp, Artificial Sequence
  SPI1 tether
gctgctaacgcaagagcaatcccaggagccctaggtttggctggtgcagttatgatgcttttatga

SEQ ID NO: 30 - protein, 21 aa, Artificial Sequence
  Synthetic construct
AANARAIPGALGLAGAVMMLL

SEQ ID NO: 31 - DNA, 156 bp, Artificial Sequence
  SPI tether
ttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 32 - protein, 51 aa, Artificial Sequence
  Synthetic construct
LTITNCPCTIEKPTSETSVSSTHDVETNSNAANARAIPGALGLAGAVMMLL

SEQ ID NO: 33 - DNA, 246 bp, Artificial Sequence
  SPI1 tether
gaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 34 - protein, 81 aa, Artificial Sequence
  Synthetic construct
EVVSEFTTYCPEPTTFVTNGATFTVTAPTTLTITNCPCTIEKPTSETSVSSTHDVETNSNAANARAIPGALGLAGAVMMLL

SEQ ID NO: 35 - DNA, 336 bp, Artificial Sequence
  SPI1 tether
attgccggtaacgatacagccacgccagcaccagagccatcatccgccgctccaatattctacaactcgactgctactgcaacacagtacgaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 36 - protein, 111 aa, Artificial Sequence
  Synthetic construct
IAGNDTATPAPEPSSAAPIFYNSTATATQYEVVSEFTTYCPEPTTFVTNGATFTVTAPTTLTITNCPCTIEKPTSETSVSSTHDVETNSNAANARAIPGALGLAGAVMMLL

SEQ ID NO: 37 - DNA, 75 bp, Artificial Sequence
  CCW12 tether
tacactggtgctgctgctaaggctttgccagctgctggtgctttgttggctggtgccgctgctttgttgttgtaa

SEQ ID NO: 38 - protein, 24 aa, Artificial Sequence
  synthetic construct
YTGAAAKALPAAGALLAGAAALLL

SEQ ID NO: 39 - DNA, 150 bp, Artificial Sequence
  CCW12 tether
actgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 40 - protein, 49 aa, Artificial Sequence
  Synthetic construct
TAAPVTSTEAPKNTTSAAPTHSVTSYTGAAAKALPAAGALLAGAAALLL

SEQ ID NO: 41 - DNA, 225 bp, Artificial Sequence
  CCW12 tether
accgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 42 - protein, 74 aa, Artificial Sequence
  Synthetic construct
TVDDVITQYTTWCPLTTEAPKNGTSTAAPVTSTEAPKNTTSAAPTHSVTSYTGAAAKALPAAGALLAGAAALLL

SEQ ID NO: 43 - DNA, 300 bp, Artificial Sequence
  CCW12 tether
gtcaccatcacttcttgtgaagaccacgtctgttctgaaactgtctccccagctttggtttccaccgctaccgtcaccgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 44 - protein, 99 aa, Artificial Sequence
  Synthetic construct
VTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 45 - DNA, 499 bp, Artificial Sequence
  Promoter of TEF2 gene
gggcgccataaccaaggtatctatagaccgccaatcagcaaactacctccgtacattcctgttgcacccacacatttatacacccagaccgcgacaaattacccataaggttgtttgtgacggcgtcgtacaagagaacgtgggaactttttaggctcaccaaaaaagaaaggaaaaatacgagttgctgacagaagcctcaagaaaaaaaaaattcttcttcgactatgctggagccagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggttttttttaaggactacttgtttttagaatatacggtcaacgaactataattaactaaac

SEQ ID NO: 46 - DNA, 500 bp, Artificial Sequence
  ADH3 terminator
tagcgtgttacgcacccaaactttttatgaaagtctttgtttataatgatgaggtttataaatatatagtggagcaaagattaatcactaaatcaagaagcagtaccagtattttttttatatcaagtagtgataatggaaatagcccaaatttggcttccgtcggcacatagcacgtttgagagacattatcaccatcaagcatcgagccgcccaaacctaactgtataagttttttcacgtttttgatttttccttgcacacttcgatattactctcacgataaaagggccgaagagaatatttttcttgaacatccagaattttaattcggagaaatttcacaagccgccgatttaagggtcctgtgttcttaataatcagcctctctcaaagcaggtaagaggcagtctttcttttaacaataggagacattcgaactaaaacatcagccccaaaaatgcgcttgaaggtcattaggatttggatttcttcctcat

SEQ ID NO: 47 - DNA, 500 bp, Artificial Sequence
  Terminator of IDP1 gene
tcgaatttacgtagcccaatctaccactttttttttcattttttaaagtgttatacttagttatgctctaggataatgaactacttttttttttttttactgttatcataaatatatataccttattgatgtttgcaaccgtcggttaattccttatcaaggttccccaagttcggatcattaccatcaatttccaacatcttcatgagttcttcttcttcattaccgtgttttagggggctgttcgcacttctaatagggctatcaccaagctgttctaattcgtccaaaagttcagtaacacgatctttatgcttcagttcgtcataatctttcaattcataaatatttacaatttcgtctacgatattaaattgcctcttgtaggtgcctatcttttccttatgctcttcattttcaccgttttcttgaaaccaaacaccgaactcactacgcatttctttcataggctcatataatacttcttttgacgtcatttg

SEQ ID NO: 48 - protein, 19 aa, Saccharomyces cerevisiae
MLLQAFLFLLAGFAAKISA

SEQ ID NO: 49 - protein, 22 aa, Artificial Sequence
  HA Tag
KDNSSTIEGRYPYDVPDYALQA

SEQ ID NO: 50 - protein, 10 aa, Artificial Sequence
  Linker
GGGGSGGGGS

SEQ ID NO: 51 - protein, 18 aa, Artificial Sequence
  Signal sequence from AGA2
MQLLRCFSIFSVIASVLA

SEQ ID NO: 52 - DNA, 1641 bp, Artificial Sequence
  FLO1 tether
  CDS (1)..(1641)
aaggacaatagctcgacgattgaaggtagatacccatacgacgttccagactacgctctgcaggctagtggtggtggtggttctggtggtggtggttctggtggtggtggttctgctagcatcagaactccaaccagtgaaggtttggttacaaccaccactgaaccatggactggtacttttacttcgacttccactgaaatgtctactgtcactggaaccaatggcttgccaactgatgaaactgtcattgttgtcaaaactccaactactgccatctcatccagtttgtcatcatcatcttcaggacaaatcaccagctctatcacgtcttcgcgtccaattattaccccattctatcctagcaatggaacttctgtgatttcttcctcagtaatttcttcctcagtcacttcttctctattcacttcttctccagtcatttcttcctcagtcatttcttcttctacaacaacctccacttctatattttctgaatcatctaaatcatccgtcattccaaccagtagttccacctctggttcttctgagagcgaaacgagttcagctggttctgtctcttcttcctcttttatctcttctgaatcatcaaaatctcctacatattcttcttcatcattaccacttgttaccagtgcgacaacaagccaggaaactgcttcttcattaccacctgctaccactacaaaaacgagcgaacaaaccactttggttaccgtgacatcctgcgagtctcatgtgtgcactgaatccatctcccctgcgattgtttccacagctactgttactgttagcggcgtcacaacagagtataccacatggtgccctatttctactacagagacaacaaagcaaaccaaagggacaacagagcaaaccacagaaacaacaaaacaaaccacggtagttacaatttcttcttgtgaatctgacgtatgctctaagactgcttctccagccattgtatctacaagcactgctactattaacggcgttactacagaatacacaacatggtgtcctatttccaccacagaatcgaggcaacaaacaacgctagttactgttacttcctgcgaatctggtgtgtgttccgaaactgcttcacctgccattgtttcgacggccacggctactgtgaatgatgttgttacggtctatcctacatggaggccacagactgcgaatgaagagtctgtcagctctaaaatgaacagtgctaccggtgagacaacaaccaatactttagctgctgaaacgactaccaatactgtagctgctgagacgattaccaatactggagctgctgagacgaaaacagtagtcacctcttcgctttcaagatctaatcacgctgaaacacagacggcttccgcgaccgatgtgattggtcacagcagtagtgttgtttctgtatccgaaactggcaacaccaagagtctaacaagttccgggttgagtactatgtcgcaacagcctcgtagcacaccagcaagcagcatggtaggatatagtacagcttctttagaaatttcaacgtatgctggcagtgccaacagcttactggccggtagtggtttaagtgtcttcattgcgtccttattgctggcaattatttaa

SEQ ID NO: 53 - protein, 546 aa, Artificial Sequence
  Synthetic Construct
KDNSSTIEGRYPYDVPDYALQASGGGGSGGGGSGGGGSASIRTPTSEGLVTTTTEPWTGTFTSTSTEMSTVTGTNGLPTDETVIVVKTPTTAISSSLSSSSSGQITSSITSSRPIITPFYPSNGTSVISSSVISSSVTSSLFTSSPVISSSVISSSTTTSTSIFSESSKSSVIPTSSSTSGSSESETSSAGSVSSSSFISSESSKSPTYSSSSLPLVTSATTSQETASSLPPATTTKTSEQTTLVTVTSCESHVCTESISPAIVSTATVTVSGVTTEYTTWCPISTTETTKQTKGTTEQTTETTKQTTVVTISSCESDVCSKTASPAIVSTSTATINGVTTEYTTWCPISTTESRQQTTLVTVTSCESGVCSETASPAIVSTATATVNDVVTVYPTWRPQTANEESVSSKMNSATGETTTNTLAAETTTNTVAAETITNTGAAETKTVVTSSLSRSNHAETQTASATDVIGHSSSVVSVSETGNTKSLTSSGLSTMSQQPRSTPASSMVGYSTASLEISTYAGSANSLLAGSGLSVFIASLLLAII

SEQ ID NO: 54 - protein, 435 aa, Thermococcus gammatolerans
KYSELEQGGVIMQAFYWDVPAGGIWWDTIRQKIPEWYDAGISAIWIPPASKGMGGAYSMGYDPYDYFDLGEFYQKGTVETRFGSKEELVNMISTAHRYGIKVIADIVINHRAGGDLEWNPYVGDYTWTDFSQVASGKYKAHYMDFHPNNYSTSDEGTFGGFPDIDHLVPFNKYWLWASDESYAAYLRSIGVDAWRFDYVKGYGAWVVKDWLSWWGGWAVGEYWDTDVNALLNWAYDSGAKVFDFPLYYKMDEAFDNKNIPALVYAIQNGGTVVSRDPFKAVTFVANHDTNIIWNKYPAYAFILTYEGQPVIFYRDYEEWLNKDKLNNLIWIHEHLAGGSTKILYYDDDELIFMREGYGDRPGLITYINLGSGWAERWVNVGSKFAGYTIHEYTGNLGGWVDRYVYYNGWVKLTAPPHDPANGYYGYSVWSYAGVG

SEQ ID NO: 55 - protein, 432 aa, Thermococcus thioreducens
ETLENGGVIMQAFYWDVPMGGIWWDTIAQKIPDWASAGISAIWIPPASKGMSGGYSMGYDPYDYFDLGEYYQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASNESYAAYLRSIGIDAWRFDYVKGYAPWVVKDWLNWWGGWAVGEYWDTNVDALLNWAYASGAKVFDFPLYYKMDEAFDNNNIPALVDALRYGQTVVSRDPFKAVTFVANHDTDIIWNKYPAYAFILTYEGQPMIFYRDYEEWLNKDRLKNLIWIHDHLAGGSTDIVYYDSDELIFVRNGYGSKPGLITYINLGSSKAGRWVYVPKFAGSCIHEYTGNLGGWVDKWVDSSGWVYLEAPAHDPANGQYGYSVWSYCGVG

SEQ ID NO: 56 - protein, 440 aa, Thermococcus eurythermalis
QPAGAAKYLELEEGGVIMQAFYWDVPSGGIWWDTIRQKIPEWYDAGISAIWIPPASKGMGGAYSMGYDPYDFFDLGEYDQKGTVETRFGSKQELVNMINTAHAYGIKVIADIVINHRAGGDLEWNPFVNDYTWTDFSKVASGKYTANYLDFHPNEVKCCDEGTFGGFPDIAHEKSWDQYWLWASNESYAAYLRSIGVDAWRFDYVKGYGAWVVKDWLDWWGGWAVGEYWDTNVDALLNWAYSSDAKVFDFPLYYKMDAAFDNKNIPALVEALKNGGTVVSRDPFKAVTFVANHDTDIIWNKYPAYAFILTYEGQPTIFYRDYEEWLNKDRLKNLIWIHDHLAGGSTDIVYYDNDELIFVRNGYGDKPGLITYINLGSSKAGRWVYVPKFAGACIHEYTGNLGGWVDKWVDSSGWVYLEAPAHDPANGYYGYSVWSYCGVG

SEQ ID NO: 57 - protein, 432 aa, Thermococcus hydrothermalis
ETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 58 - protein, 434 aa, Pyrococcus furiosus
KYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 59 - protein, 436 aa, Thermococcus gammatolerans
MKYSELEQGGVIMQAFYWDVPAGGIWWDTIRQKIPEWYDAGISAIWIPPASKGMGGAYSMGYDPYDYFDLGEFYQKGTVETRFGSKEELVNMISTAHRYGIKVIADIVINHRAGGDLEWNPYVGDYTWTDFSQVASGKYKAHYMDFHPNNYSTSDEGTFGGFPDIDHLVPFNKYWLWASDESYAAYLRSIGVDAWRFDYVKGYGAWVVKDWLSWWGGWAVGEYWDTDVNALLNWAYDSGAKVFDFPLYYKMDEAFDNKNIPALVYAIQNGGTVVSRDPFKAVTFVANHDTNIIWNKYPAYAFILTYEGQPVIFYRDYEEWLNKDKLNNLIWIHEHLAGGSTKILYYDDDELIFMREGYGDRPGLITYINLGSGWAERWVNVGSKFAGYTIHEYTGNLGGWVDRYVYYNGWVKLTAPPHDPANGYYGYSVWSYAGVG

SEQ ID NO: 60 - protein, 433 aa, Thermococcus thioreducens
METLENGGVIMQAFYWDVPMGGIWWDTIAQKIPDWASAGISAIWIPPASKGMSGGYSMGYDPYDYFDLGEYYQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASNESYAAYLRSIGIDAWRFDYVKGYAPWVVKDWLNWWGGWAVGEYWDTNVDALLNWAYASGAKVFDFPLYYKMDEAFDNNNIPALVDALRYGQTVVSRDPFKAVTFVANHDTDIIWNKYPAYAFILTYEGQPMIFYRDYEEWLNKDRLKNLIWIHDHLAGGSTDIVYYDSDELIFVRNGYGSKPGLITYINLGSSKAGRWVYVPKFAGSCIHEYTGNLGGWVDKWVDSSGWVYLEAPAHDPANGQYGYSVWSYCGVG

SEQ ID NO: 61 - protein, 441 aa, Thermococcus eurythermalis
MQPAGAAKYLELEEGGVIMQAFYWDVPSGGIWWDTIRQKIPEWYDAGISAIWIPPASKGMGGAYSMGYDPYDFFDLGEYDQKGTVETRFGSKQELVNMINTAHAYGIKVIADIVINHRAGGDLEWNPFVNDYTWTDFSKVASGKYTANYLDFHPNEVKCCDEGTFGGFPDIAHEKSWDQYWLWASNESYAAYLRSIGVDAWRFDYVKGYGAWVVKDWLDWWGGWAVGEYWDTNVDALLNWAYSSDAKVFDFPLYYKMDAAFDNKNIPALVEALKNGGTVVSRDPFKAVTFVANHDTDIIWNKYPAYAFILTYEGQPTIFYRDYEEWLNKDRLKNLIWIHDHLAGGSTDIVYYDNDELIFVRNGYGDKPGLITYINLGSSKAGRWVYVPKFAGACIHEYTGNLGGWVDKWVDSSGWVYLEAPAHDPANGYYGYSVWSYCGVG

SEQ ID NO: 62 - protein, 433 aa, Thermococcus hydrothermalis
METLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 63 - protein, 436 aa, Pyrococcus furiosus
MAKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 64 - protein, 435 aa, Pyrococcus furiosus
MKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 65 - protein, 435 aa, Pyrococcus furiosus
MKYSELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 66 - protein, 436 aa, Pyrococcus furiosus
MSKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 67 - protein, 435 aa, Thermococcus hydrothermalis
MKYETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 68 - protein, 435 aa, Thermococcus hydrothermalis
MKYSELENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 69 - protein, 434 aa, Thermococcus hydrothermalis
MSETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 70 - protein, 21 aa, Saccharomyces cerevisiae
MRFPSIFTAVLFAASSALAAP

SEQ ID NO: 71 - protein, 17 aa, Artificial Sequence
  Linker
SGGGGSGGGGSGGGGSA

SEQ ID NO: 72 - protein, 115 aa, Artificial Sequence
  Synthetic construct
AANVTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 73 - protein, 108 aa, Artificial Sequence [sequence omitted]

SEQ ID NO: 74 - protein, 16 aa, Artificial Sequence
  Amino acid linker
  VARIANT (3)...(16): Any one or all of amino acids 3-16 can either be present or absent.
GSGSGSGSGSGSGSGS

SEQ ID NO: 75 - protein, 24 aa, Artificial Sequence
  Amino acid linker
  VARIANT (4)...(24): Any one or all of amino acids 4-24 can either be present or absent.
GGSGGSGGSGGSGGSGGSGGSGGS

SEQ ID NO: 76 - protein, 32 aa, Artificial Sequence
  Amino acid linker
  VARIANT (5)...(32): any one or all of amino acids 5-32 can either be present or absent.
GGGSGGGSGGGSGGGSGGGSGGGSGGGSGGGS

SEQ ID NO: 77 - protein, 40 aa, Artificial Sequence; identical to SEQ ID NO: 24
  Amino acid linker
  VARIANT (6)...(40): any one or all of amino acids 6-40 can either be present or absent.

SEQ ID NO: 78 - protein, 32 aa, Artificial Sequence
  Amino acid linker
  VARIANT (5)...(32): any one or all of amino acids 5-32 can either be present or absent.
GGSGGGSGGGSGGGSGGGSGGGSGGGSGGGSG

SEQ ID NO: 79 - protein, 32 aa, Artificial Sequence
  Amino acid linker
  VARIANT (5)...(32): any one or all of amino acids any one or all of amino acids 5-32 can either be  present or absent.
GSATGSATGSATGSATGSATGSATGSATGSAT

(19) United States
(12) Patent Application Publication
(10) Pub. No.: US 2022/0267818 A1
(43) Pub. Date: Aug. 25, 2022

(54) YEAST EXPRESSING THERMOSTABLE ALPHA-AMYLASES FOR HYDROLYSIS OF STARCH

(71) Applicant: Lallemand Hungary Liquidity Management LLC, Budapest (HU)

(72) Inventors: Aaron Argyros, Lebanon, NH (US); Charles F. Rice, Plainfield, NH (US)

(21) Appl. No.: 16/980,111

(22) PCT Filed: Mar. 13, 2019
(86) PCT No.: PCT/IB2019/052050

§ 371 Date: Sep. 11, 2020

Related U.S. Application Data
(60) Provisional application No. 62/642,542, filed on Mar. 13, 2018.

(51) Int. Cl. C12P 19/14; C12N 15/62; C12N 9/28; C12P 19/12; C07K 14/39

(52) U.S. Cl. CPC C12P 19/14; C12N 15/625; C12N 9/2417; C12Y 302/01001; C12P 19/12; C07K 14/39; C07K 2319/03; C07K 2319/02

(57) ABSTRACT

The present disclosure concerns the recombinant expression of thermostable alpha-amylases in a yeast host cell, compositions and yeast products made from the recombinant yeast host cells as well as the use of the thermostable alpha-amylase for hydrolyzing starch and ultimately making a fermentation product.

### TECHNICAL FIELD

The present disclosure relates to polypeptides having thermostable alpha-amylase activity for hydrolysis of starch (including raw starch), more specifically the present disclosure relates to the expression of such polypeptides in recombinant yeast cells.

### BACKGROUND

Yeast cells, such as Saccharomyces cerevisiae, are the primary biocatalyst used in the commercial production of fuel ethanol. This organism is proficient in fermenting glucose to ethanol, often to concentrations greater than 20% w/v. However, yeasts such as S. cerevisiae lack the ability to hydrolyze polysaccharides and therefore require the exogenous addition of purified enzymes to convert complex sugars to simpler molecules, such as glucose. For example, in the United States, the primary source of fuel ethanol is corn starch, which, regardless of the mashing process, requires the exogenous addition of both alpha-amylase and glucoamylase. The cost of the purified enzymes range from $0.02-0.04 per gallon, which at 14 billion gallons of ethanol produced each year, represents a substantial cost savings opportunity for producers if they could reduce their enzyme dose.

In a broad sense, one major fermentation process in the corn ethanol industry is by using liquefied corn mash. In the mash process, corn is both thermally and enzymatically liquefied using alpha-amylases prior to fermentation in order to break down long chain starch polymers into smaller dextrins. The mash is then cooled and inoculated with S. cerevisiae along with the exogenous addition of purified glucoamylase, an exo-acting enzyme which will further break down the dextrin into utilizable glucose molecules.

During the liquefaction of starch, conventional processes will heat a starch-containing medium to effect gelatinization of the starch, thereby disrupting the crystalline structure and improving the accessibility of the starch molecules to enzymatic action. Alpha-amylase being the primary activity of importance for reducing viscosity and initiating hydrolysis of the starch, must be capable of tolerating temperatures as high as 85-90° C. In some processes, a jet cooker is used to further increase shearing of starch molecules, raising the temperatures to over 105° C., which often denatures early alpha-amylase additions resulting in the need for additional doses once temperatures are decreased back to 85° C. to finish hydrolysis. Subsequently, the mash is cooled and pumped into the fermenter where the pH is lower below 5.0 and yeast inoculated for ethanol production. However, the pH of the liquefaction is typically between 5.5-6.2, the optimum for most alpha-amylases.

It would be desirable to be provided with improved alpha-amylases for the liquefaction of starch. It would further be desirable to reduce the need for external/exogenous enzyme addition during the liquefaction process. It would further be desirable to simplify the process for the liquefaction of starch, for example, to reduce the number of steps, the time, the complexity and/or the costs associated therewith. It would be desirable to have an alpha-amylase that could exhibit adequate activity after the heat treatment, especially during the jet cooking process, thereby further reducing the exogenous enzyme additions. It would therefore be desirable to have an alpha-amylase that is highly active at the lower pH conditions to avoid process costs of pH changes.

### SUMMARY

The present disclosure concerns thermostable alpha-amylase which can be used to liquefy starch prior to the fermentation process. The alpha-amylase enzymes of the present disclosure exhibit thermo-tolerance, e.g. activity at temperatures above 60° C.

According to a first aspect, the present disclosure provides a recombinant yeast host cell comprising a heterologous nucleic acid molecule encoding a heterologous polypeptide having thermostable alpha-amylase activity. The heterologous polypeptide comprises the amino acid sequence of SEQ ID NO: 54, a variant or a fragment thereof; SEQ ID NO: 55, a variant or a fragment thereof; SEQ ID NO: 56, a variant or a fragment thereof; SEQ ID NO: 57, a variant or a fragment thereof, or SEQ ID NO: 58, a variant or a fragment thereof. In a specific embodiment, the heterologous polypeptide comprises the amino acid sequence of SEQ ID NO: 57, a variant or a fragment thereof. In further embodiments, the recombinant yeast host cell can express a first heterologous polypeptide (such as, for example, the heterologous polypeptide having the amino acid sequence of SEQ ID NO: 57, a variant thereof or a fragment thereof) and a second heterologous polypeptide (such as, for example, the heterologous polypeptide having the amino acid sequence of SEQ ID NO: 38, a variant thereof or a fragment thereof). In an embodiment, the heterologous polypeptide further comprises a signal sequence (which can be, in some embodiments, native to the heterologous polypeptide). In some embodiments, the heterologous polypeptide comprises the amino acid sequence of: SEQ ID NO: 1, a variant or a fragment thereof; SEQ ID NO: 2, a variant or a fragment thereof; SEQ ID NO: 3, a variant or a fragment thereof; SEQ ID NO: 4, a variant or a fragment thereof; or SEQ ID NO: 5, a variant or a fragment thereof. In another embodiment, the signal sequence is heterologous to the heterologous polypeptide. For example, the heterologous signal sequence can be derived from the invertase protein and have, in some further embodiments, the amino acid sequence of SEQ ID NO: 48, a variant thereof or a fragment thereof. In an embodiment, the heterologous polypeptide has alpha-amylase activity at a temperature of at least 60° C., 65° C., 70° C., 75° C., 80° C., 85° C., 90° C., 95° C. or 99° C.

In another embodiment, the heterologous nucleic acid molecule is operatively associated with an heterologous promoter sequence for allowing the expression of the polypeptide during propagation. In still another embodiment, the heterologous polypeptide is a secreted polypeptide. In yet another embodiment, the heterologous polypeptide is a cell-associated polypeptide such as intracellular polypeptide or a membrane-associated polypeptide (a tethered heterologous polypeptide for example). In another embodiment, the heterologous polypeptide is a polypeptide of formula (I) or (II):

(NH₂)SS-HP-L-TT(COOH) (I)

(NH₂)SS-TT-L-HP(COOH) (II)

wherein SS is present or absent and is an heterologous signal sequence (which is removed by cleavage during the secretion of the heterologous polypeptide); HP is the heterologous polypeptide having alpha-amylase activity; L is present or absent and is an amino acid linker; TT is present or absent and is an amino acid tethering moiety for associating the heterologous polypeptide to a cell wall of the recombinant yeast host cell; (NH₂) indicates the amino terminus of the polypeptide; (COOH) indicates the carboxyl terminus of the polypeptide; and “-” is an amide linkage. In an embodiment, TT is present. In yet another embodiment, TT can be modified by a post-translation mechanism to have a glycosylphosphatidylinositol (GPI) anchor. For example, TT can be from a SED1 protein, a SPI1 protein, a CCW12 protein, a CWP2 protein, a TIR1 protein, a PST1 protein, a combination of a AGA1 and a AGA2 protein, a variant thereof or a fragment thereof. In an embodiment, TT is from the SPI1 protein and comprises an amino acid sequence set forth in any one of SEQ ID NOs: 30, 32, 34 and 36. In still another embodiment, TT is from the CCW12 protein and comprises an amino acid sequence set forth in any one of SEQ ID NOs: 38, 40, 42 and 44. In yet another embodiment, L is present and comprises an amino acid sequence as set forth in any one of SEQ ID NOs: 22 to 28.

In some embodiments, the heterologous polypeptide is an intracellular polypeptide. For example, the heterologous polypeptide can lack a signal sequence. In some embodiments, the heterologous polypeptide has the amino acid sequence of (i) any one of SEQ ID NO: 59 to 63 in which the first amino acid residue after the methionine residue at position 1 has been removed; (ii) any one of SEQ ID NO: 59 to 63 in which the first two consecutive amino acid residues after the methionine residue at position 1 have been removed; and/or (iii) any one of SEQ ID NO: 59 to 63 in which at least one lysine, tyrosine, serine, or glutamic acid residue has been added after the methionine residue at position 1. In some embodiments, the heterologous polypeptide has the amino acid sequence of SEQ ID NO: 64, a variant or a fragment thereof; SEQ ID NO: 65, a variant or a fragment thereof; SEQ ID NO: 66, a variant or a fragment thereof; SEQ ID NO: 67, a variant or a fragment thereof; SEQ ID NO: 68, a variant or a fragment thereof; or SEQ ID NO: 69, a variant or a fragment thereof.

In yet another embodiment, the heterologous nucleic acid molecule further encodes a chimeric protein comprising the heterologous polypeptide fused to a starch binding domain. In yet another embodiment, the heterologous nucleic molecule further encodes an heterologous glucoamylase. In an embodiment, the recombinant yeast host cell from the genus Saccharomyces. In still another embodiment, the recombinant yeast host cell from the species Saccharomyces cerevisiae.

According to a second aspect, the present application provides a purified, isolated and/or recombinant polypeptide having thermostable alpha-amylase activity obtained from a recombinant yeast host cell as described herein. In some embodiments, the purified polypeptide is a chimeric polypeptide of formula (III) or (IV):

(NH₂)SS-HP-L-TT(COOH) (III)

(NH₂)SS-TT-L-HP(COOH) (IV)

wherein SS is present or absent and is an heterologous signal sequence (which is removed by cleavage during the secretion of the heterologous polypeptide); HP is the heterologous polypeptide having alpha-amylase activity; L is present or absent and is an amino acid linker; TT is an amino acid tethering moiety for associating the chimeric polypeptide to a cell wall of the recombinant yeast host cell; (NH₂) indicates the amino terminus of the polypeptide; (COOH) indicates the carboxyl terminus of the polypeptide; and “-” is an amide linkage.

According to a third aspect, the present disclosure provides a composition comprising the recombinant yeast host cell described herein, the purified polypeptide described herein and at least one of a glucoamylase or starch.

According to a fourth aspect, the present disclosure provides a yeast product made from the recombinant yeast host cell described or comprising the purified polypeptide described herein. In an embodiment, the yeast product is an inactivated yeast product such as, for example, a yeast extract.

According to a fifth aspect, the present disclosure provides a process for hydrolyzing starch, the process comprising contacting the recombinant yeast host cell described herein, the purified polypeptide described herein or the yeast product described herein with a medium comprising starch. In an embodiment, the medium comprises raw starch. In another embodiment, the medium is derived from corn. In some embodiments, the process comprises adding the recombinant yeast host cell described herein, the purified polypeptide described herein, the composition described herein or the yeast product described herein to a liquefaction medium and, in additional embodiments, heating the liquefaction medium to obtain a liquefied medium. In still another embodiment, the process can be used for making a fermentation product (for example from the liquefied medium). In such embodiment, the process can further comprise fermenting the liquefied medium with a first yeast cell to obtain the fermented product. In yet another embodiment, the fermentation product is ethanol.

### BRIEF DESCRIPTION OF THE DRAWINGS

FIG. 1 provides the alpha-amylase activity on raw starch at 85° C. (measured as the absorbance at 540 nm) of various recombinant yeast strains (A, B, C, D and E, described in Table 1) expressing alpha-amylases according to some embodiments of the present disclosure.

FIG. 2 shows the thermal tolerance of the supernatant of different yeast-made alpha-amylases measured after a heat treatment of 30 min at various temperatures followed by a starch assay at 85° C. for 60 min. Results are shown as the absorbance measured at 540 nm in function of temperature of the heat treatment and yeast strain (●=Thermococcus thioreducens (A), X=Thermococcus gammatolerans (B), ▪=Pyrococcus furiosus (C), ▴=Thermococcus hydrothermalis (D) and ♦=Thermococcus eurythermalis (E), described in Table 1).

FIG. 3 is a schematic diagram of an expression cassette according to an embodiment of the present disclosure.

FIG. 4 shows the alpha-amylase activity on raw starch at 85° C. associated with various recombinant yeast strains expressing tethered alpha-amylases having different combinations of alpha-amylases (from left to right SEQ ID NO: 56, SEQ ID NO: 57 and SEQ ID NO: 58) in the presence of different tethering moieties (from left to right for each alpha-amylase SED1, TIR1, CWP2, CCW12, SPI1) or in a free “secreted” form, according to some embodiments of the present disclosure. Results are shown as a measurement of reducing sugars using the DNS assay on raw starch at 85° C. (the absorbance measured at 540 nm) in function of the different alpha-amylases and tethering moieties tested. The condition “secreted” refers to the secreted form (e.g., not tethered) of the alpha-amylase.

FIG. 5 shows the alpha-amylase activity associated with the cells of yeast strains expressing various chimeric proteins comprising an alpha-amylase derived from P. furiosus (SEQ ID NO: 5) in combination with different tethering moieties derived from the SPI1 protein or associated truncations (M15774, M15771, M15777, M15772 and M15222, described in Table 2). Results are shown as a measurement of reducing sugars using the DNS assay and the absorbance at 540 nm in function of the yeast strain.

FIG. 6 shows the alpha-amylase activity associated with cells of yeast strains expressing various chimeric proteins comprising an alpha-amylases derived from T. hydrothermalis (SEQ ID NO: 4) in combination with different tethering moieties derived from the CCW12 protein or associated truncations (M15773, M15776, M16251, M15775 and M15215, described in Table 2). Results are shown as a measurement of reducing sugars using the DNS assay and the absorbance at 540 nm in function of the yeast strain.

FIG. 7 shows the alpha-amylase activity associated with the cells of yeast strains expressing various chimeric proteins comprising an alpha-amylase derived from T. hydrothermalis (SEQ ID NO: 4) in combination with a tethering moiety derived from the CCW12 protein and different linkers (M15785, M15786, M15782, M16252, M16221 and M16222, described in Table 2). Results are shown as a measurement of reducing sugars using the DNS assay and the absorbance at 540 nm in function of the yeast strain.

FIG. 8 shows the alpha-amylase activity associated with the cells of yeast strains expressing various chimeric proteins comprising an alpha-amylase derived from P. furiosus (SEQ ID NO: 5), a tethering moiety derived from the SPI1 protein and different linkers (M15784, M15778, M15779, M15787, M15780, M15788 and M15783, described in Table 2). Results are shown as a measurement of reducing sugars using the DNS assay and the absorbance at 540 nm in function of the yeast strain.

FIG. 9 shows a dextrose equivalent profile associated with the M15958 strain during a laboratory scale fermentation. Results are shown as the percentage of dextrose equivalent in function of time (minutes).

FIG. 10 shows the alpha-amylase activity associated with the cells expressing variants of the P. furiosus alpha-amylase (M16450, M19211, M15900, M19246, M19247, and M19249 described in Table 5). Results are sown as a measurement of reducing sugars using the DNS assay (measured as the absorbance at 540 nm) in a function of the yeast strain.

FIG. 11 shows the alpha-amylase activity of variants of the T. hydrothermalis alpha-amylase (M16450, M19211, M15899, M19251, M19253, and M19256, described in Table 5). Results are sown as a measurement of reducing sugars using the DNS assay (measured as the absorbance at 540 nm) in a function of the yeast strain.

FIG. 12 shows the alpha-amylase activity of different strains (M10474 (parent), M14964 (expressing a secreted T. eurythermalis alpha-amylase), M14965 (expressing a secreted T. hydrothermalis alpha-amylase), M14966 (expressing a secreted P. furiosus alpha-amylase), M15591 (expressing a secreted T. thioreducens), or M15592 (expressing a secreted T. gammatolerans), described in Table 3) in a 1 gram small scale liquefaction. The results are shown as the absorbance read at 540 nm (to determine the reducing sugars measured using the DNS assay) in function of the yeast strain.

FIG. 13 shows the alpha-amylase activity of different strains (M10474 (parent), M16789 (expressing a tethered T. hydrothermalis alpha-amylase), M16790 (expressing a tethered T. thioreducens alpha-amylase), M16791 (expressing a tethered P. furiosus alpha-amylase), of M16792 (expressing a tethered T. gammatolerans alpha-amylase), described in Table 3) in a 1 gram small scale liquefaction. The results are shown as the absorbance read at 540 nm (to determine the reducing sugars measured using the DNS assay) in function of the yeast strain.

FIG. 14 shows the endpoint dextrose equivalent of a lab-scale liquefaction of 0.045% g dry cell weight (DCW)/g solids of inactivated alpha-amylase expressing yeast strain, M16449, without any enzyme added (0.045% M16449); 0.045% g DCW/g solids of inactivated alpha-amylase expressing yeast strain, M16449, along with 0.005% commercial alpha-amylase enzyme added (0.045% M16449+0.005% commercial alpha-amylase enzyme); 0.045% g DCW/g solids of inactivated alpha-amylase expressing yeast strain, M16449, along with, 0.0025% commercial alpha-amylase enzyme added (0.045% M16449+0.0025% commercial alpha-amylase enzyme); or a full dose (100%) of the commercial alpha-amylase enzyme (0.02% w/w). Results are shown as % dextrose equivalent (Y axis) as a function of the liquefaction conditions (X axis).

FIG. 15 shows fermentation performance of the M2390 strain, in a 32% solids fermentation using lab-scale liquefactions dosed with: of 0.045% g dry cell weight (DCW)/g solids of inactivated alpha-amylase expressing yeast strain, M16449, without any enzyme added (0.045% M16449); 0.045% g DCW/g solids of inactivated alpha-amylase expressing yeast strain, M16449, along with 0.005% commercial alpha-amylase enzyme added (0.045% M16449+0.005% commercial alpha-amylase enzyme); 0.045% g DCW/g solids of inactivated alpha-amylase expressing yeast strain, M16449, along with, 0.0025% commercial alpha-amylase enzyme added (0.045% M16449+0.0025% commercial alpha-amylase enzyme); or a full dose (100%) of the commercial alpha-amylase enzyme (0.02% w/w) (X axis). Results are shown as ethanol concentration (Y axis, in g/L) as a function of the liquefaction conditions (X-axis).

FIG. 16 shows the torque trend profile of lab-scale liquefactions containing: commercial alpha-amylaseenzyme #1 dosed at 100% (0.02% w/w) (light dashed line); commercial alpha-amylase enzyme #2 dosed at 100% (0.02% w/w) (dark dashed line); 0.045% g DCW/g solids additions of inactivated alpha-amylase expressing yeast, M19211 (▪); 0.03% g DCW/g solids additions of inactivated alpha-amylase expressing yeast, M19211 (●), 0.03% g DCW/g solids additions of inactivated alpha-amylase expressing yeast, M19211, along with a 50% (0.01%) dose of commercial alpha-amylase enzyme #2 (♦); or 0.03% gDCW/g solids additions of inactivated alpha-amylase expressing yeast, M19211, along with a 25% (0.005%) dose of commercial alpha-amylase enzyme #2 (▴). Results are shown as torque trends in Newton Centimeters (left Y axis) as a function of time (X axis, h:mm:ss).

FIG. 17 shows the endpoint dextrose equivalent of a lab-scale liquefaction containing: commercial alpha-amylase enzyme #1 dosed at 100% (0.02% w/w, commercial alpha-amylase enzyme #1); commercial alpha-amylase enzyme #2 dosed at 100% (0.02% w/w, commercial alpha-amylase enzyme #2); 0.045% g DCW/g solids additions of inactivated alpha-amylase expressing yeast M19211 (0.045% DCW M19211); 0.03% g DCW/g solids additions of inactivated alpha-amylase expressing yeast M19211 (0.03% DCW M191211), 0.03% DCW/g solids additions of inactivated alpha-amylase expressing yeast M19211, along with a 25% (0.01%) dose of commercial alpha-amylase enzyme #2 (0.03% DCW M19211+0.005% commercial alpha-amylase enzyme #2); or 0.03% g DCW/g solids additions of inactivated alpha-amylase expressing yeast M19211, along with a 50% (0.01%) dose of commercial alpha-amylase enzyme #2 (0.03 DCW 19211+0.01% commercial alpha-amylase enzyme #2) The data is reported as % dextrose equivalent (Y axis) as a function of the liquefaction conditions (X axis).

FIG. 18 shows the torque trend profile of lab-scale liquefactions containing: commercial alpha-amylases enzyme #1 dosed at 100% (0.02% w/w) (dark dashed line); commercial alpha-amylases enzyme #2 dosed at 100% (0.02% w/w) (light dashed line); autolysized strain M19211 dosed at 0.03% g DCW/g solids additions of inactivated alpha-amylase expressing yeast, with a 25% (0.005%) dose of commercial alpha-amylase enzyme #1 (●); bead beaten or milled strain M19211 dosed at 0.03% g DCW/g solids additions of inactivated alpha-amylase expressing yeast, with a 25% (0.005%) dose of commercial alpha-amylase enzyme #1 (▴); or high pressure homogenized strain M19211 dosed at 0.03% g DCW/g solids additions of inactivated alpha-amylase expressing yeast, with a 25% (0.005%) dose of commercial alpha-amylase enzyme #1 (▪). Results shown as torque trends in Newton Centimeters (Y axis) as a function of time (X-axis, h:mm:ss).

FIG. 19 shows the endpoint dextrose equivalent of a lab-scale liquefaction containing: autolysized strain M19211 dosed at 0.03% g DCW/g solids additions of inactivated alpha-amylase expressing yeast, with a 25% (0.005%) dose of commercial alpha-amylase enzyme #1 (autolysis 0.003% DCW M19211+0.0005% commercial alpha-amylase enzyme #1); bead beaten or milled strain M19211 dosed at 0.03% g DCW/g solids additions of inactivated alpha-amylase expressing yeast, with a 25% (0.005%) dose of commercial alpha-amylase enzyme #1 (bead milled 0.003% DCW M19211+0.005% commercial alpha-amylase enzyme #1); high pressure homogenized strain M19211 dosed at 0.03% g DCW/g solids additions of inactivated alpha-amylase expressing yeast, with a 25% (0.005%) dose of commercial alpha-amylase enzyme #1 (high pressure homogenization 0.03% DCW M19211+0.005% commercial alpha-amylase enzyme #1); commercial alpha-amylase enzyme #1 dosed at 100% (0.02% w/w, commercial alpha-amylase enzyme #1); or commercial alpha-amylase enzyme #2 dosed at 100% (0.02% w/w, commercial alpha-amylase enzyme #2). Results are shown as % dextrose equivalent (Y axis) as a function of the liquefaction conditions (X axis).

FIG. 20 shows the dextrose equivalent profile of a 1 g mini-liquefaction hydrolyzed with various M19211 inactivation methods: cream unwashed, cream washed, bead milled unwashed, high pressure homogenized unwashed, high pressure homogenized washed, instant dry yeast (IDY) unwashed, IDY washed, YPD unprocessed, and YPD bead beaten. Results are shown as % dextrose equivalent (Y axis) as a function of inactivation methods (X axis).

FIG. 21 shows the torque trend profile of lab-scale liquefactions containing: commercial alpha-amylase enzyme #1 dosed at 100% (0.02% w/w) (dark dashed line); commercial alpha-amylase enzyme #2 dosed at 100% (0.02% w/w) (light dashed line); 0.03% g DCW/g solids additions YPD propped, bead milled inactivated alpha-amylase expressing yeast, M19211, along with a 25% dose of commercial alpha-amylase enzyme #1 (0.005%) (▴); 0.03% g DCW/g solids additions alpha-amylase expressing yeast, M19211, inactivated by washed high pressure homogenization, along with a 25% dose of commercial alpha-amylase enzyme #1 (0.005%) (●); or 0.03% g DCW/g solids additions alpha-amylase expressing yeast, M19211, inactivated by unwashed high pressure homogenization, along with a 25% dose of commercial alpha-amylase enzyme #1 (0.005%) (▪). Results are shown as torque trends in Newton Centimeters (Y axis) as a function of time (X axis, h:mm:ss).

FIG. 22 shows the endpoint dextrose equivalent of a lab-scale liquefaction containing: 0.03% g DCW/g solids additions YPD propped, bead milled inactivated alpha-amylase expressing yeast, M19211, along with a 25% dose of commercial alpha-amylase enzyme #1 (0.005%, YPD propped, bead milled); 0.03% g DCW/g solids additions alpha-amylase expressing yeast, M19211, inactivated by washed high pressure homogenization, along with a 25% dose of commercial alpha-amylase enzyme #1 (0.005%, washed high pressure homogenization); 0.03% g DCW/g solids additions alpha-amylase expressing yeast, M19211, inactivated by unwashed high pressure homogenization, along with a 25% dose of commercial alpha-amylase enzyme #1 (0.005%, unwashed high pressure homogenization); commercial alpha-amylase enzyme #1 dosed at 100% (0.02% w/w); commercial alpha-amylase enzyme #2 dosed at 100% (0.02% w/w) (X axis). Results shown as % dextrose equivalent (Y axis) as a function of the liquefaction conditions (X axis).

FIG. 23 shows the potential ethanol obtained by using the M2390 strain, in a 33% solids fermentation using lab-scale liquefactions dosed with: commercial alpha-amylase enzyme #2 dosed at 100% (0.02% w/w, commercial alpha-amylase enzyme #2); commercial alpha-amylase enzyme #1 dosed at 100% (0.02% w/w, commercial alpha-amylase enzyme #1); 0.03% g DCW/g solids additions YPD propped, bead milled inactivated alpha-amylase expressing yeast, M19211, along with a 25% dose of commercial alpha-amylase enzyme #1 (0.005%, YPD propped); 0.03% g DCW/g solids additions alpha-amylase expressing yeast, M19211, inactivated by washed high pressure homogenization, along with a 25% dose of commercial alpha-amylase enzyme #1 (0.005%, washed high pressure homogenization); or 0.03% g DCW/g solids additions alpha-amylase expressing yeast, M19211, inactivated by unwashed high pressure homogenization, along with a 25% dose of commercial alpha-amylase enzyme #1 (0.005%, unwashed high pressure homogenization). Results are shown as potential ethanol concentration (left Y axis, gray bars, in g/L) as a function of the liquefaction conditions.

### DETAILED DESCRIPTION

The present disclosure relates to polypeptides having thermostable alpha-amylase activity to facilitate starch liquefaction (for example for improving the hydrolysis of starch, including the hydrolysis of raw starch). The use of such polypeptides, in some embodiments, reduces the amount of or avoids the use of adscititious/external/exogenous enzyme (such as purified alpha-amylase preparation) used during the liquefaction of starch, such as in the production of ethanol. The polypeptides having thermostable alpha-amylase activity of the present disclosure are intended to be expressed in recombinant yeast host cells. The polypeptides can be provided from a recombinant yeast host cell or a product derived from the recombinant yeast host cell.

The polypeptides of the present disclosure have alpha-amylase activity. Polypeptides having alpha-amylase activity (also referred to as alpha amylases; EC 3.2.1.1) are endo-acting enzymes capable of hydrolyzing starch to maltose and maltodextrins. Some alpha-amylases are calcium metalloenzymes which are unable to function in the absence of calcium. However, archaeal alpha-amylases as those described herein do not have a calcium dependency. By acting at random locations along the starch chain, alpha-amylases break down long-chain carbohydrates, ultimately yielding, maltodextrins, maltotriose, maltose and smaller chain dextrins from amylose, or maltose, glucose and “limit dextrin” from amylopectin. Alpha-amylase activity can be determined by various ways by the person skilled in the art. For example, the alpha-amylase activity of a polypeptide can be determined directly by measuring the amount of reducing sugars generated by the polypeptide in an assay in which raw (corn) starch (such as, for example, raw starch) is used as the starting material. The alpha-amylase activity of a polypeptide can be measured indirectly by measuring the amount of reducing sugars generated by the polypeptide in an assay in which starch (raw or gelatinized) is used as the starting material.

In the context of the present disclosure, a polypeptide having thermostable alpha-amylase activity means that the polypeptides exhibit relative alpha-amylase activity of at least 10%, 20%, 30%, 40%, 50%, 60%, 70%, 80%, 90%, 95%, 96%, 97%, 98% or 99% of the alpha-amylase activity of the amino acid of any one of SEQ ID NOs: 1 to 5 and 54 to 69 after being subjected to elevated temperatures. In some embodiments, the elevated temperatures correspond to a temperature range encountered during the liquefaction process which can be a temperature of at least 60° C., 65° C., 70° C., 75° C., 80° C., 85° C., 90° C., 95° C., or 99° C. In yet another embodiment, the elevated temperatures are maintained for at least 5, 10, 15, 20, 25, 30, 35, 40, 45, 50, 55, 60, 65, 70, 75, 80, 85, 90, 95, 100, 105, 110, 115, 120 minutes or more prior to determining the alpha-amylase activity of the polypeptide.

Polypeptides having the thermostable alpha-amylase activity are expressed from one or more heterologous nucleic acid molecules in one or more recombinant host cell. In some embodiments, the recombinant yeast host cell is capable of fermenting glucose to ethanol (such as, for example, in a recombinant yeast host cell). The thermostable alpha-amylase polypeptides of the present disclosure can break-down starch to smaller molecular weight molecules, such as oligosaccharides and/or dextrins. The polypeptides having thermostable alpha-amylase activity are heterologous with respect to the recombinant yeast host cell expressing them. As used herein, the term “heterologous” when used in reference to a nucleic acid molecule (such as a promoter, a terminator or a coding sequence) or a polypeptide refers to a nucleic acid molecule or a protein that is not natively found in the recombinant host cell. “Heterologous” also includes a native coding region/promoter/terminator, or portion thereof, that was introduced into the source organism in a form and/or at a location that is different from the corresponding native gene, e.g., not in its natural location in the organism's genome. The heterologous nucleic acid molecule is purposively introduced into the recombinant host cell. For example, a heterologous element could be derived from a different strain of host cell, or from an organism of a different taxonomic group (e.g., different domain, kingdom, phylum, class, order, family genus, or species, or any subgroup within one of these classifications).

In an embodiment, the recombinant yeast host cells of the present disclosure may express the polypeptides having thermostable amylase activity during propagation so as to increase the concentration/amount of the thermostable alpha-amylase prior to its introduction in the liquefaction medium and/or the liquefied (fermentation) medium. In an embodiment, the heterologous polypeptides having thermostable alpha-amylase activity can be added to the liquefaction medium prior to and/or during the heating step to gelatinize starch (e.g., liquefaction step). In some embodiments, the heterologous polypeptides having thermostable alpha-amylase activity can also be added after the heating step to complete the liquefaction of starch. These embodiments seek to increase process efficiency, expression of the recombinant polypeptide may reduce the need for exogenous alpha-amylase enzymes.

During liquefaction, a substrate including starch may be heated and/or maintained at temperatures of greater than about 60° C. Gelatinization of the starch present in corn usually begins at around about 70° C. to 75° C. In a typical corn ethanol process, the temperature can be raised and held at a temperature around 80° C. to 85° C. and can even reach a temperature of 105° C. (when a jet-cooker is used). At temperatures between 70° C. and 105° C., starch molecules tend to gelatinize, improving its availability for enzymatic breakdown. However, at such temperatures, conventional alpha-amylase enzymes usually exhibit a decrease in alpha-amylase activity. For example, alpha-amylase enzymes may be denatured at higher temperatures. As a result, conventional processes for liquefying starch require large amounts of exogenous enzyme (e.g., exogenous alpha-amylases, including, but not limited to, maltogenic alpha-amylases) to ensure proper liquefaction which may require large operating costs to purchase sufficient exogenous enzyme In contrast to the conventional alpha-amylases, polypeptides with thermostable alpha-amylase activity, such as the heterologous polypeptides of the present disclosure, may exhibit alpha-amylase activity at higher temperatures. The addition of such heterologous polypeptides or the recombinant host yeast cells expressing such heterologous polypeptides to the liquefaction medium may reduce the amount of exogenous alpha-amylase used during liquefaction, or simplify liquefaction process such that cooling of the substrate material is reduced or eliminated. In some embodiments, the recombinant yeast host cells expressing the heterologous polypeptides having thermostable alpha-amylase activity and/or the polypeptides having thermostable alpha-amylase activity may, for example, be used to effect enzymatic break-down of starch while the starch is heated, thereby simplifying the overall process.

### Recombinant Host Cells

The polypeptides described herein can independently be provided in a purified form (derived from the recombinant yeast host cell described herein) or expressed in a recombinant host cell. The recombinant host cell thus includes at least one genetic modification. In the context of the present disclosure, when recombinant yeast cell is qualified has “having a genetic modification” or as being “genetically engineered”, it is understood to mean that it has been manipulated to either add at least one or more heterologous or exogenous nucleic acid residue and/or remove at least one endogenous (or native) nucleic acid residue. The genetic manipulations did not occur in nature and is the results of in vitro manipulations of the recombinant host cell. When the genetic modification is the addition of an heterologous nucleic acid molecule, such addition can be made once or multiple times at the same or different integration sites. When the genetic modification is the modification of an endogenous nucleic acid molecule, it can be made in one or both copies of the targeted gene.

When expressed in a recombinant host, the heterologous polypeptides described herein are encoded on one or more heterologous nucleic acid molecule. The term “heterologous” when used in reference to a nucleic acid molecule (such as a promoter or a coding sequence) refers to a nucleic acid molecule that is not natively found in the recombinant host cell. “Heterologous” also includes a native coding region, or portion thereof, that is introduced into the source organism in a form that is different from the corresponding native gene, e.g., not in its natural location in the organism's genome. The heterologous nucleic acid molecule is purposively introduced into the recombinant host cell. Thus, for example, an heterologous element could be derived from a different strain of host cell, or from an organism of a different taxonomic group (e.g., different domain, kingdom, phylum, class, order, family genus, or species, or any subgroup within one of these classifications).

When an heterologous nucleic acid molecule is present in the recombinant host cell, it can be integrated in the host cell's genome. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the yeast host cell's genome. Alternatively, the heterologous nucleic acid molecule can be independently replicating from the yeast's genome. In such embodiment, the nucleic acid molecule can be stable and self-replicating.

In the context of the present disclosure, the recombinant host cell can be a recombinant yeast host cell. Suitable recombinant yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, S. cerevisiae, S. bulderi, S. barnetti, S. exiguus, S. uvarum, S. diastaticus, S. boulardii, K. lactis, K. marxianus or K. fragilis. In some embodiments, the recombinant yeast host cell is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In some additional embodiments, the recombinant yeast host cell is from Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and/or Schwanniomyces occidentalis. In some embodiments, the recombinant host cell can be an oleaginous yeast cell. For example, the recombinant oleaginous yeast host cell can be from the genera Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the recombinant host cell can be an oleaginous microalgae host cell (e.g., for example, from the genera Thraustochytrium or Schizochytrium). In an embodiment, the recombinant yeast host cell is from the genus Saccharomyces and, in some embodiments, from the species Saccharomyces cerevisiae.

One of the genetic modifications that can be introduced into the recombinant host is the introduction of one or more of an heterologous nucleic acid molecule encoding an heterologous polypeptide (such as, for example, the polypeptides having thermostable alpha-amylase activity as described herein). In an embodiment, the recombinant host can be modified to express two distinct thermostable alpha-amylases (or more) which can be encoded on a single heterologous nucleic acid molecule or on distinct heterologous nucleic acid molecules.

In some embodiments, the recombinant host cell comprises a genetic modification (e.g., a heterologous nucleic acid molecule) allowing the recombinant expression of the polypeptide having thermostable alpha-amylase activity. In such embodiment, a heterologous nucleic acid molecule encoding the polypeptide having thermostable alpha-amylase activity can be introduced in the recombinant host to express the polypeptide having thermostable alpha-amylase activity. The expression of the polypeptide having thermostable alpha-amylase activity can be constitutive or induced.

In an embodiment, the recombinant yeast host cell can include a further heterologous nucleic acid molecule including a coding sequence for an heterologous glucoamylase. Alternatively or in combination, the recombinant yeast host cell can be used in combination with an heterologous glucoamylase (either provided in a recombinant form or in a purified form).

### Heterologous Polypeptides Having Thermostable Alpha-amylase Activity

In the context of the present disclosure, the heterologous polypeptides having thermostable alpha-amylase activity (which can be expressed intracellularly, in a tethered form or a secreted form as indicated therein) can be derived from a bacterial, eukaryotic or archaeal cell. In some embodiments, for example, the polypeptides or portions thereof are derived from the family Thermococcaceae and, in some instances, from the genus Thermococcus or Pyrococcus. In some embodiments, the polypeptides or portions thereof are derived from a cell derived from Thermococcus eurythermalis, Thermococcus hydrothermalis, Pyrococcus furiosus, Thermococcus thioreducens, and Thermococcus gammatolerans.

The heterologous polypeptide of the present disclosure can be expressed inside the recombinant yeast host cell, e.g., intracellularly. The polypeptides of the present disclosure can be modified to remove, if any, signal peptide sequences present in the native amino acid sequence of the polypeptide to allow for an intracellular expression. In some embodiments, the polypeptides of the present disclosure can be modified to replace the signal sequence with a N-terminus modification (for example methionine at the N-terminus) to allow for an intracellular expression (as explained herein for N-terminus variants of the heterologous polypeptide). In some embodiments, the intracellularly expressed heterologous polypeptide includes a thermostable alpha-amylase polypeptide derived from a Pyrococcus furiosus alpha-amylase as set forth in any one of SEQ ID NOs: 58 or 63 to 66, a Thermococcus thioreducensalpha-amylase as set forth in SEQ ID NO: 55 or 60, a Thermococcus eurythermalis alpha-amylase as set forth in SEQ ID NO: 56 or 61, a Thermococcus hydrothermalis alpha-amylase as set forth in any one of SEQ ID NOs: 57, 62 or 67 to 69, a Thermococcus gammatolerans alpha-amylase as set forth in SEQ ID NO: 54 or 59, or a variant or a fragment thereof. In an embodiment, the intracellularly expressed heterologous polypeptide comprises the amino acid sequence of SEQ ID NO: 57, a variant or a fragment thereof (which is present in the amino acid sequence of SEQ ID NO: 62 and 67 to 69). In another embodiment, the intracellularly expressed heterologous polypeptide has the amino acid sequence of SEQ ID NO: 58, a variant thereof or a fragment thereof (which is present in the amino acid sequence of SEQ ID NO: 63 to 66). In still another embodiment, the intracellularly expressed heterologous polypeptide comprises the amino acid sequence of SEQ ID NO: 57, a variant or a fragment thereof (which is present in the amino acid sequence of SEQ ID NO: 62 and 67 to 69) and the intracellularly expressed heterologous polypeptide has the amino acid sequence of SEQ ID NO: 58, a variant thereof or a fragment thereof (which is present in the amino acid sequence of SEQ ID NO: 63 to 66).

In some embodiments, the heterologous polypepide includes a thermostable alpha-amylase polypeptide from Pyrococcus furiosus (GenBank Accession #WP_014835153.1) as set forth in SEQ ID NO: 5, Thermococcus thioreducens (GenBank Accession #WP_055428342.1) as set forth in SEQ ID NO: 2, Thermococcus eurythermalis (GenBank Accession #WP_050002265.1) as set forth in SEQ ID NO: 3, Thermococcus hydrothermalis (GenBank Accession #AAC97877.1) as set forth in SEQ ID NO: 4 and/or Thermococcus gammatolerans (GenBank Accession #ACS32724.1) as set forth in SEQ ID NO: 1, or a variant or a fragment thereof. In an embodiment, the heterologous polypeptide has the amino acid sequence of SEQ ID NO: 4, a variant thereof or a fragment. In an embodiment, the heterologous polypeptide has the amino acid sequence of SEQ ID NO: 5, a variant thereof or a fragment thereof.

Still in the context of the present disclosure, the heterologous polypeptides include variants of the alpha-amylases polypeptides of any one of SEQ ID NOs: 1 to 5 and 54 to 69 (also referred to herein as thermostable alpha-amylase variants). A variant comprises at least one amino acid difference (substitution or addition) when compared to the amino acid sequence of the thermostable alpha-amylase polypeptide of any one of SEQ ID NOs: 1 to 5 and 54 to 69. The thermostable alpha-amylase variants exhibit thermostable alpha-amylase activity. In an embodiment, the variant thermostable alpha-amylase exhibits at least 50%, 60%, 70%, 80%, 90%, 95%, 96%, 97%, 98% or 99% of the wild-type thermostable alpha-amylase activity having the amino acid of any one of SEQ ID NOs: 1 to 5 and 54 to 69 after having been exposed to elevated temperatures (such as, for example, a temperature of about 60° C., 65° C., 70° C., 75° C., 80° C., 85° C., 90° C., 95° C., 99° C., or more). The thermostable alpha-amylase variants also have at least 70%, 80%, 85%, 90%, 95%, 96%, 97%, 98% or 99% identity to the amino acid sequence of any one of SEQ ID NOs: 1 to 5 and 54 to 69. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant thermostable alpha-amylases described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant thermostable alpha-amylase can be also be a conservative variant or an allelic variant. As used herein, a conservative variant refers to alterations in the amino acid sequence that do not adversely affect the biological functions of the thermostable alpha-amylase (e.g., hydrolysis of starch). A substitution, insertion or deletion is said to adversely affect the protein when the altered sequence prevents or disrupts a biological function associated with the thermostable alpha-amylase (e.g., the hydrolysis of starch into maltose and maltodextrins). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the thermostable alpha-amylase.

In the context of the present disclosure, the intracellularly expressed heterologous polypeptide can be modified at the N-terminus to provide variant heterologous polypeptides. If the heterologous polypeptide includes a native signal sequence, it can be removed to allow the intracellular expression of the heterologous polypeptide. In some embodiments, the intracellularly expressed heterologous polypeptide is selected to have or is modified to have a first methionine residue (e.g., a methionine residue at position 1). In some embodiments, 1, 2, 3, 4, 5, 6, 7, 8, 9, 10, or more consecutive amino acid residues are removed from the native sequence and optionally at the N-terminus, after the first methionine. The removed amino acid residues can be positioned right next (e.g., following) to the first methionine. Alternatively or in combination, 1, 2, 3, 4, 5, 6, 7, 8, 9, 10, or more consecutive amino acid residues are added starting at the second position from the N-terminus, following the first methionine. In some embodiments, 1, 2, 3, 4, 5, 6, 7, 8, 9, 10, or more amino acid residues are removed starting at the second position from the N-terminus, following the first methionine. In some embodiments, both 1, 2, 3, 4, 5, 6, 7, 8, 9, 10, or more amino acid residues are removed and 1, 2, 3, 4, 5, 6, 7, 8, 9, 10, or more amino acid residues are added, starting at the second position from the N-terminus, following the first methionine. In some specific embodiments, a single amino acid residue (e.g., at position 2) is removed, following the first methionine. In such embodiment, one or more consecutive amino acid residues can be added at the site of the deletion. In some alternative embodiments, two consecutive amino acid residues (e.g., at positions 2 and 3) are removed, following the first methionine. In such embodiment, one, two or more consecutive amino acid residues can be added at the site of the deletion. In some additional embodiments, three consecutive amino acid residues (e.g., at positions 2 to 4) are removed, following the first methionine. In such embodiment, one, two or three consecutive amino acid residues can be added at the site of the deletion. In some further embodiments, four consecutive amino acid residues (e.g., at positions 2 to 5) are removed following the first methionine. In some embodiments, one, two, three or four consecutive amino acid residues are added at the site of the deletion. In some embodiments, the modifications are made to intracellularly express heterologous polypeptide having a first methionine. In an embodiment, the variant heterologous polypeptide has the amino acid sequence of SEQ ID NO: 59, a variant thereof or a fragment thereof. In another embodiment, the variant heterologous polypeptide has the amino acid sequence of SEQ ID NO: 60, a variant thereof or a fragment thereof. In a further embodiment, the variant heterologous polypeptide has the amino acid sequence of SEQ ID NO: 61, a variant thereof or a fragment thereof. In yet another embodiment, the variant heterologous polypeptide has the amino acid sequence of SEQ ID NO: 63, a variant thereof or a fragment thereof. In still a further embodiment, the variant heterologous polypeptide has the amino acid sequence of SEQ ID NO: 63, a variant thereof or a fragment thereof.

In some embodiments, the heterologous polypeptide having the amino acid sequence of SEQ ID NO: 58 is modified to include a methionine residue at position 1. In such embodiment, the heterologous polypeptide can further be modified remove the first amino acid residue (e.g., alanine) after the first methionine to provide the heterologous polypeptide of SEQ ID NO: 64. In some embodiments, the heterologous polypeptide is still further modified to include at least one of lysine residue, a tyrosine residue or a serine residue to provide, in some embodiments, the heterologous polypeptide having the amino acid sequence of SEQ ID NO: 65 or 66.

In some embodiments, the heterologous polypeptide having the amino acid sequence of SEQ ID NO: 57 is modified to include a methionine at position 1. In such embodiment, the heterologous polypeptide is modified to add at least one a lysine residue, a tyrosine residue or a serine residue after the first methionine to provide, for example, the heterologous polypeptide of SEQ ID NO: 67 or 69. Alternatively, the heterologous polypeptide can be further modified to remove at least one (a glutamic acid residue) or two (a glutamic acid residue and a threonine residue) amino acid residues after the first methionine. In such embodiment, the heterologous polypeptide can still further be modified to add at least one a lysine residue, a tyrosine residue or a serine residue after the first methionine to provide, for example, the heterologous polypeptide of SEQ ID NO: 68.

The present disclosure also provide fragments of the thermostable alpha-amylase activity polypeptides and thermostable alpha-amylase variants described herein. A fragment comprises at least one less amino acid residue when compared to the amino acid sequence of the (wild-type) thermostable alpha-amylase polypeptide or variant (described herein) and still possess the enzymatic activity of the full-length alpha-amylase (at the same temperature as the full-length alpha-amylase). For example, a fragment can correspond to the thermostable alpha-amylase or a variant thereof described herein to which the signal peptide sequence has been removed. In an embodiment, the fragment of the thermostable alpha-amylase or the variant thereof exhibits at least 10%, 20%, 30%, 40%, 50%, 60%, 70%, 80%, 90%, 95%, 96%, 97%, 98% or 99% of the alpha-amylase activity of the full-length amino acid of any one of SEQ ID NOs: 1 to 5 and 54 to 69 after having been exposed to elevated temperatures (e.g., such as, for example, a temperature of about 60° C., 65° C., 70° C., 75° C., 80° C., 85° C., 90° C., 95° C., 99° C., or more). The thermostable alpha-amylase fragments can also have at least 10%, 20%, 30%, 40%, 50%, 60%, 70%, 80%, 85%, 90%, 95%, 96%, 97%, 98% or 99% identity to the amino acid sequence of any one of SEQ ID NOs: 1 to 5 and 54 to 69. The fragment can be, for example, a truncation of one or more amino acid residues at the amino-terminus, the carboxy terminus or both terminus of the thermostable alpha-amylase polypeptide or variant. Alternatively or in combination, the fragment can be generated from removing one or more internal amino acid residues. In an embodiment, the thermostable alpha-amylase fragment has at least 100, 150, 200, 250, 300, 350, 400, 450 or more consecutive amino acids of the thermostable alpha-amylase polypeptide or the variant.

The heterologous polypeptide of the present disclosure can be expressed for secretion outside the recombinant yeast host cell. In some embodiments, the polypeptide includes one or a combination of signal peptide sequence(s) allowing the transport of the polypeptide outside the yeast host cell's wall. The signal sequence can simply be added to the polypeptide or replace the signal peptide sequence already present in the protein from which the thermostable alpha-amylase activity portion is derived. The signal sequence can be native or heterologous to the protein from which the thermostable alpha-amylase activity portion is derived. In some embodiments, one or more signal sequences can be used. In some embodiments, the one or more signal sequences are cleaved once the heterologous polypeptide is secreted. In some embodiments, the signal sequence is from the invertase protein (and can have, for example, the amino acid sequence of SEQ ID NO: 48, be a variant of the amino acid sequence of SEQ ID NO: 48 or be a fragment of the amino acid sequence of SEQ ID NO: 48); the AGA2 protein (and can have, for example, the amino acid sequence of SEQ ID NO: 51, be a variant of the amino acid sequence of SEQ ID NO: 51 or be a fragment of the amino acid sequence of SEQ ID NO: 51); or the α-mating factor protein (and can have, for example, the amino acid sequence of SEQ ID NO: 70, be a variant of the amino acid sequence of SEQ ID NO: 70 or be a fragment of the amino acid sequence of SEQ ID NO: 70). In the context of the present disclosure, the expression “functional variant of a signal sequence” refers to a nucleic acid sequence that has been substituted in at least one nucleic acid position when compared to the native signal sequence which retain the ability to direct the expression of the polypeptide outside the cell. In the context of the present disclosure, the expression “functional fragment of a signal sequence” refers to a shorter nucleic acid sequence than the native signal sequence which retain the ability to direct the expression of the polypeptide outside the cell.

### Chimeric Heterologous Polypeptides having Thermostable Alpha-Amylase Activity

The heterologous polypeptide of the present disclosure can be provided in a chimeric form and fused to a starch binding domain. As used herein, “a starch binding domain” is a polypeptide sequence having affinity to starch. For example, the starch binding domain can be from a polypeptide having glucoamylase activity from the genus Aspergillus, in some instances, from the species Aspergillus niger, in further instances, from an Aspergillus niger G1 glucoamylase (and have, for example, the amino acid sequence of SEQ ID NO: 76, be a variant of the amino acid sequence of SEQ ID NO: 76 or be a fragment of the amino acid sequence of SEQ ID NO: 76). The starch binding domain can be located at the amino or carboxy terminus of the heterologous polypeptides of the present disclosure.

In some embodiments, the chimeric polypeptide having the thermostable alpha-amylase moiety is a polypeptide of formula (I) or (II):

(NH₂)SS-TT-L-HP(COOH) (I)

(NH₂)SS-HP-L-TT(COOH) (II)

wherein:
HP is the heterologous polypeptide having thermostable alpha-amylase activity;
L is present or absent and is an amino acid linker;
TT is present or absent and is an amino acid tethering moiety for associating the polypeptide to a cell wall or cell membrane of the recombinant yeast host cell;
SS is present or absent and is a signal sequence moiety;
(NH₂) indicates the amino terminus of the polypeptide;
(COOH) indicates the carboxyl terminus of the polypeptide; and
“-” is an amide linkage.

In other embodiments, the polypeptides of the present disclosure can be secreted. When the polypeptides are secreted, they are transported to outside of the cell. In such embodiments, the polypeptides having thermostable alpha-amylase activity of formula (I) and (II) have a SS moiety but lack a TT moiety.

In some embodiments, the polypeptides of the present disclosure remain physically associated with the recombinant yeast host cell when secreted. In an embodiment, at least one portion (usually at least one terminus) of the polypeptide is bound, covalently, non-covalently and/or electrostatically for example, to cell wall (and in some embodiments to the cytoplasmic membrane). For example, the polypeptide can be modified to bear one or more transmembrane domains, to have one or more lipid modifications (myristoylation, palmitoylation, farnesylation and/or prenylation), to interact with one or more membrane-associated protein and/or to interactions with the cellular lipid rafts. While the polypeptide may not be directly bound to the cell membrane or cell wall (e.g., such as when binding occurs via a tethering moiety), due to the polypeptide's physical association with the cell, it is nonetheless considered a “cell-associated” polypeptide according to the present disclosure. In some embodiments, the polypeptides, when expressed, include one or more signal sequences for facilitating the secretion of the polypeptides. The signal sequences may be cleaved during the secretion of the polypeptides to an extracellular space and consequently is absent from the secreted form of the chimeric protein.

In some embodiments, the heterologous polypeptide of the present disclosure is “cell-associated” to the recombinant yeast host cell because it is designed to be expressed and remain physically associated with the recombinant yeast host cells. In an embodiment, the polypeptide can be expressed inside the recombinant yeast host cell (intracellularly). In such embodiment, the polypeptide does not need to be associated to the recombinant yeast host cell's wall. When the polypeptide is intended to be expressed intracellularly, its signal sequence, if present in the native sequence, can be deleted to allow intracellular expression.

In some embodiments, the heterologous polypeptide can be expressed to be located at and associated to the cell wall of the recombinant yeast host cell. In some embodiments, the polypeptide is expressed to be located at and associated to the external surface of the cell wall of the host cell. Recombinant yeast host cells all have a cell wall (which includes a cytoplasmic membrane) defining the intracellular (e.g., internally-facing the nucleus) and extracellular (e.g., externally-facing) environments. The polypeptide can be located at (and in some embodiments, physically associated to) the external face of the recombinant yeast host's cell wall and, in further embodiments, to the external face of the recombinant yeast host's cytoplasmic membrane. In the context of the present disclosure, the expression “associated to the external face of the cell wall/cytoplasmic membrane of the recombinant yeast host cell” refers to the ability of the polypeptide to physically integrate (in a covalent or non-covalent fashion), at least in part, in the cell wall (and in some embodiments in the cytoplasmic membrane) of the recombinant yeast host cell. The physical integration can be attributed to the presence of, for example, a transmembrane domain on the polypeptide, a domain capable of interacting with a cytoplasmic membrane protein on the polypeptide, a post-translational modification made to the polypeptide (e.g., lipidation), etc. In some embodiments, the polypeptides having thermostable activity which are associated to the membrane of the recombinant yeast host cell of formula (I) or (II) have a SS moiety and a TT moiety, with an optional L moiety.

In some embodiments, the heterologous polypeptides of the present disclosure can be expressed inside the recombinant yeast host cell, e.g., intracellularly. In such embodiments, the polypeptides having thermostable activity of formula (I) or (II) lack the SS moiety, the L moiety and the TT moiety. The polypeptides of the present disclosure expressed intracellularly can be modified to remove, if any, signal peptide sequences present in the native amino acid sequence of the polypeptide to allow for an intracellular expression.

As indicated above, in some embodiments, the polypeptide includes one or a combination of signal peptide sequence(s) allowing the transport of the polypeptide outside the yeast host cell's wall. The signal sequence can simply be added to the polypeptide or replace the signal peptide sequence already present in the protein from which the thermostable alpha-amylase activity portion is derived. The signal sequence can be native or heterologous to the protein from which the thermostable alpha-amylase activity portion is derived. In some embodiments, one or more signal sequences can be used. In some embodiments, the one or more signal sequences are cleaved once the polypeptide is secreted. In some embodiments, the signal sequence is from the invertase protein (and can have, for example, the amino acid sequence of SEQ ID NO: 48, be a variant of the amino acid sequence of SEQ ID NO: 48 or be a fragment of the amino acid sequence of SEQ ID NO: 48); the AGA2 protein (and can have, for example, the amino acid sequence of SEQ ID NO: 51, be a variant of the amino acid sequence of SEQ ID NO: 51 or be a fragment of the amino acid sequence of SEQ ID NO: 51); or the α-Mating factor protein (and can have, for example, the amino acid sequence of SEQ ID NO: 70, be a variant of the amino acid sequence of SEQ ID NO: 70 or be a fragment of the amino acid sequence of SEQ ID NO: 70). In the context of the present disclosure, the expression “functional variant of a signal sequence” refers to a nucleic acid sequence that has been substituted in at least one nucleic acid position when compared to the native signal sequence which retain the ability to direct the expression of the polypeptide outside the cell. In the context of the present disclosure, the expression “functional fragment of a signal sequence” refers to a shorter nucleic acid sequence than the native signal sequence which retain the ability to direct the expression of the polypeptide outside the cell.

As indicated above, in some embodiments, the polypeptides include an amino acid tethering moiety (TT) which will provide or increase attachment to the cell wall of the recombinant host cell. In such embodiment, the chimeric polypeptide will be considered “tethered”. TT may increase or provide cell association to some polypeptides because they exhibit insufficient intrinsic cell association or simply lack intrinsic cell association. In some embodiments, the amino acid tethering moiety of the chimeric polypeptide is neutral with respect to the biological activity of the thermostable alpha-amylase activity portion, e.g., does not interfere with the biological activity. In some embodiments, the association of the amino acid tethering moiety with the thermostable alpha-amylase activity portion can increase the biological activity of thermostable alpha-amylase activity portion (when compared to the non-tethered, “free” form). Various tethering amino acid moieties are known to the art and can be used in the chimeric proteins of the present disclosure. The tethering moiety can be a transmembrane domain found on another protein and allow the polypeptide to have a transmembrane domain. TT may be endogenous or exogenous to the host cell. In some embodiments, TT is endogenous to the host cell.

In some embodiments where TT is present, the polypeptide is a chimeric polypeptide of formula (III) or (IV):

(NH₂)SS-HP-L-TT(COOH) (III)

(NH₂)SS-TT-L-HP(COOH) (IV)

wherein:
SS is present or absent and is an heterologous signal sequence;
HP is the heterologous polypeptide having alpha-amylase activity;
L is present or absent and is an amino acid linker;
TT is an amino acid tethering moiety for associating the chimeric polypeptide to a cell wall of the recombinant yeast host cell;
(NH₂) indicates the amino terminus of the polypeptide;
(COOH) indicates the carboxyl terminus of the polypeptide; and
“-” is an amide linkage.

In some embodiments, the polypeptides, when expressed, include one or more signal sequences for facilitating the secretion of the polypeptides. The signal sequences may be cleaved during the secretion of the polypeptides to an extracellular space and consequently is absent from the secreted form of the chimeric protein. The signal sequence can simply be added to the polypeptide or replace the signal peptide sequence already present in the protein from which the thermostable alpha-amylase activity portion is derived. The signal sequence can be native or heterologous to the protein from which the thermostable alpha-amylase activity portion is derived. In some embodiments, one or more signal sequences can be used. In some embodiments, the one or more signal sequences are cleaved once the polypeptide is secreted. In some embodiments, the signal sequence is from the invertase protein (and can have, for example, the amino acid sequence of SEQ ID NO: 48, be a variant of the amino acid sequence of SEQ ID NO: 48 or be a fragment of the amino acid sequence of SEQ ID NO: 48); the AGA2 protein (and can have, for example, the amino acid sequence of SEQ ID NO: 51, be a variant of the amino acid sequence of SEQ ID NO: 51 or be a fragment of the amino acid sequence of SEQ ID NO: 51); or the α-Mating factor protein (and can have, for example, the amino acid sequence of SEQ ID NO: 70, be a variant of the amino acid sequence of SEQ ID NO: 70 or be a fragment of the amino acid sequence of SEQ ID NO: 70).

In some embodiments, TT is derived from a cell surface protein, such as a glycosylphosphotidylinositol (GPI) associated anchor protein. GPI anchors are glycolipids attached to the terminus of a protein (and in some embodiments, to the carboxyl terminus of a protein) which allows the anchoring of the protein to the cytoplasmic membrane of the cell membrane. Tethering amino acid moieties capable of providing a GPI anchor include, but are not limited to those associated with/derived from a SED1 protein (having, for example, the amino acid sequence of SEQ ID NO: 7, a variant thereof or a fragment thereof), a SPI1 protein (having, for example, the amino acid sequence of SEQ ID NO: 9, a variant thereof or a fragment thereof), a CCW12 protein (having, for example, the amino acid sequence of SEQ ID NO: 11, a variant thereof or a fragment thereof), a CWP2 protein (having, for example, the amino acid sequence of SEQ ID NO: 13, a variant thereof or a fragment thereof), a TIR1 protein (having, for example, the amino acid sequence of SEQ ID NO: 15, a variant thereof or a fragment thereof), a PST1 protein (having, for example, the amino acid sequence of SEQ ID NO: 17, a variant thereof or a fragment thereof) or a combination of a AGA1 and a AGA2 protein (having, for example, the amino acid sequence of SEQ ID NO: 19, a variant thereof or a fragment thereof or having, for example, the amino acid sequence of SEQ ID NO: 21, a variant thereof or a fragment thereof).

In some embodiments, TT can comprise a transmembrane domain, a variant or a fragment thereof. For example, the tethering moiety can be derived from the FLO1 protein (having, for example, the amino acid sequence of SEQ ID NO: 53, a variant thereof or a fragment thereof or being encoded by the nucleic acid sequence of SEQ ID NO: 52).

Still in the context of the present disclosure, TT includes variants of the tethering moieties, such as, for example, variants of SEQ ID NOs: 7, 9, 11, 13, 15, 17, 19, 21 and 53 (also referred to herein as TT variants). A variant comprises at least one amino acid difference (substitution or addition) when compared to the amino acid sequence of the original tethering moiety and is capable locating a polypeptide to the membrane of the yeast cell. The TT variants exhibit cell wall anchoring activity. In an embodiment, the TT variant exhibits at least 50%, 60%, 70%, 80%, 90%, 95%, 96%, 97%, 98% or 99% of the cell wall anchoring activity of the amino acid of any one of SEQ ID NOs: 7, 9, 11, 13, 15, 17, 19, 21 and 53. The TT variants also have at least 70%, 80%, 85%, 90%, 95%, 96%, 97%, 98% or 99% identity to the amino acid sequence of any one of SEQ ID NOs: 7, 9, 11, 13, 15, 17, 19, 21 and 53. In some embodiments, the variant of SEQ ID NO: 9 is an amino acid of any one of SEQ ID NOs: 30, 32, 34, or 36. In some embodiments, the variant of SEQ ID NO: 11 is an amino acid of any one of SEQ ID NOs: 38, 40, 42, or 44.

The TT variants described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A TT variant can be also be a conservative variant or an allelic variant.

The present disclosure also provide fragments of TT and TT variants described herein. A fragment comprises at least one less amino acid residue when compared to the amino acid sequence of the TT polypeptide or variant and still possess the cell wall anchoring activity of the full-length TT portion. In an embodiment, the TT fragment exhibits at least 50%, 60%, 70%, 80%, 90%, 95%, 96%, 97%, 98% or 99% of the cell wall anchoring activity of the amino acid of any one of SEQ ID NOs: 7, 9, 11, 13, 15, 17, 19 and 21. The TT fragments can also have at least 70%, 80%, 85%, 90%, 95%, 96%, 97%, 98% or 99% identity to the amino acid sequence of any one of SEQ ID NO: 6 to 13. The TT fragment can be, for example, a truncation of one or more amino acid residues at the amino-terminus, the carboxy terminus or both terminus of the thermostable alpha-amylase polypeptide or variant. Alternatively or in combination, the fragment can be generated from removing one or more internal amino acid residues. In an embodiment, the TT fragment has at least 10, 20, 30, 40, 50, 60, 70, 80, 90, 100, 150, 200, 250, 300, 350, 400, 450, 500, 550, 600, 650 or more consecutive amino acids of the TT portion polypeptide or the variant.

In some embodiments, the TT is a fragment of a SPI1 protein. The fragment of the SPI1 protein comprises less than 129 amino acid consecutive residues of the amino acid sequence of SEQ ID NO: 8. For example, the TT fragment is from the SPI1 protein and can comprise at least 10, 20, 21, 30, 40, 50, 51, 60, 70, 80, 81, 90, 100, 110, 111 or 120 consecutive amino acid residues from the amino acid sequence of SEQ ID NO: 8. In yet another embodiment, the TT is from the SPI1 protein can comprise or consist essentially of the amino acid sequence set forth in any one of SEQ ID NOs: 30, 32, 34 and 36.

In some embodiments, the TT is a fragment of a CCW12 protein. The fragment of the CCW12 protein comprises less than 112 amino acid consecutive residues of the amino acid sequence of SEQ ID NO: 10. For example, the TT fragment from the CCW12 protein can comprise at least 10, 20, 24, 30, 40, 49, 50, 60, 70, 74, 80, 90, 99, 100 or 110 consecutive amino acid residues from the amino acid sequence of SEQ ID NO: 10. In yet another embodiment, the TT is from the CCW12 protein and can comprise or consist essentially of the amino acid sequence set forth in any one of SEQ ID NOs: 38, 40, 42 and 44.

In embodiments in which the amino acid linker (L) is absent from the polypeptides of formula (I) and (II), the tethering amino acid moiety is directly associated with the heterologous protein. In the chimeras of formula (I), this means that the carboxyl terminus of the heterologous polypeptide moiety is directly associated (with an amide linkage) to the amino terminus of the tethering amino acid moiety. In the chimeras of formula (II), this means that the carboxyl terminus of the tethering amino acid moiety is directly associated (with an amide linkage) to the amino terminus of the heterologous protein.

In some embodiments, the presence of an amino acid linker (L) is desirable either to provide, for example, some flexibility between the heterologous protein moiety and the tethering amino acid moiety or to facilitate the construction of the heterologous nucleic acid molecule. As used in the present disclosure, the “amino acid linker” or “L” refer to a stretch of one or more amino acids separating the thermostable alpha-amylase activity portion HP and the amino acid tethering moiety TT (e.g., indirectly linking the thermostable alpha-amylase activity portion HP to the amino acid tethering moiety TT). It is preferred that the amino acid linker be neutral, e.g., does not interfere with the biological activity of the heterologous protein nor with the biological activity of the amino acid tethering moiety. In some embodiments, the amino acid linker L can increase the biological activity of the thermostable alpha-amylase activity portion and/or of the amino acid tethering moiety. In instances in which the linker (L) is present in the chimeras of formula (I), its amino end is associated (with an amide linkage) to the carboxyl end of the heterologous protein moiety and its carboxyl end is associated (with an amide linkage) to the amino end of the amino acid tethering moiety. In instances in which the linker (L) is present in the chimeras of formula (II), its amino end is associated (with an amide linkage) to the carboxyl end of the amino acid tethering moiety and its carboxyl end is associated (with an amide linkage) to the amino end of the heterologous protein moiety. Various amino acid linkers exist and include, without limitations, (GS)ₙ; (GGS)ₙ; (GGGS)ₙ; (GGGGS)ₙ; (GGSG)ₙ; (GSAT)ₙ, wherein n= is an integer between 1 to 8 (or more). In an embodiment, the amino acid linker L is (GGGGS)ₙ (also referred to as G₄S) and, in still further embodiments, the amino acid linker L comprises more than one G₄S motifs. In some embodiments, L is chosen from: (G4S)₃ (SEQ ID NO: 22), (G)₈ (SEQ ID NO: 23), (G₄S)₈ (SEQ ID NO: 24), GSAGSAAGSGEF (SEQ ID NO: 25), (EAAK)₃ (SEQ ID NO: 26), (AP)₁₀ (SEQ ID NO: 27) and A(EAAAK)₄ALEA(EAAAK)₄A (SEQ ID NO: 28). In some embodiments, the linker also includes one or more HA tag (SEQ ID NO: 49).

### Nucleic Acid Molecules for Expressing the Heterologous Polypeptides

In some embodiments, the nucleic acid molecules encoding the heterologous polypeptides, fragments or variants that can be introduced into the recombinant host cells are codon-optimized with respect to the intended recipient recombinant host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules of the present disclosure comprise a coding region for the heterologous polypeptide. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The nucleic acid molecules described herein can comprise transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced in the host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

In the heterologous nucleic acid molecule described herein, the promoter and the nucleic acid molecule coding for the heterologous polypeptide are operatively linked to one another. In the context of the present disclosure, the expressions “operatively linked” or “operatively associated” refers to fact that the promoter is physically associated to the nucleotide acid molecule coding for the polypeptide in a manner that allows, under certain conditions, for expression of the peptide from the nucleic acid molecule. In an embodiment, the promoter can be located upstream (5′) of the nucleic acid sequence coding for the heterologous protein. In still another embodiment, the promoter can be located downstream (3′) of the nucleic acid sequence coding for the heterologous polypeptide. In the context of the present disclosure, one or more than one promoter can be included in the nucleic acid molecule. When more than one promoter is included in the nucleic acid molecule, each of the promoters is operatively linked to the nucleic acid sequence coding for the polypeptide. The promoters can be located, in view of the nucleic acid molecule coding for the polypeptide, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be heterologous to the nucleic acid molecule encoding the heterologous polypeptide. The promoter can be heterologous or derived from a strain being from the same genus or species as the recombinant host cell. In an embodiment, the promoter is derived from the same genus or species of the yeast host cell and the polypeptide is derived from different genera that the host cell. One or more promoters can be used to allow the expression of the polypeptides in the recombinant yeast host cell.

In some embodiments, the host is a facultative anaerobe, such as S. cerevisiae. For facultative anaerobes, cells tend to propagate or ferment depending on the availability of oxygen. In a fermentation process, yeast cells are generally allowed to propagate before fermentation is conducted. In some embodiments, the promoter preferentially initiates transcription during a propagation phase such that the polypeptides are expressed during the propagation phase. As used in the context of the present disclosure, the expression “propagation phase” refers to an expansion phase of a commercial process in which the yeasts are propagated under aerobic conditions to maximize the conversion of a substrate into biomass. In some instances, the propagated biomass can be used in a following fermenting step (e.g. under anaerobic conditions) to maximize the production of one or more desired metabolites.

In the context of the present disclosure, the promoter or the combination of promoters present in the heterologous nucleic acid is capable of allowing the expression of the polypeptide during the propagation phase of the recombinant yeast host cell. This will allow the accumulation of the polypeptide associated with the recombinant yeast host cell prior to any subsequent use, for example in liquefaction or fermentation. In some embodiments, the promoter allows the expression of the polypeptide during the propagation phase.

The expression of the polypeptides during the propagation phase may provide sufficient expression such that the polypeptide or the recombinant yeast cells may be added during the liquefaction of starch, thereby providing yeast cells with sufficient nutrients to undergo metabolic pro The promoters can be native or heterologous to the heterologous gene encoding the heterologous protein. The promoters that can be included in the heterologous nucleic acid molecule can be constitutive or inducible promoters. Inducible promoters include, but are not limited to glucose-regulated promoters (e.g., the promoter of the hxt7 gene (referred to as hxt7p), a functional variant or a functional fragment thereof; the promoter of the ctt1 gene (referred to as ctt1p), a functional variant or a functional fragment thereof; the promoter of the glo1 gene (referred to as glo1p), a functional variant or a functional fragment thereof; the promoter of the ygp1 gene (referred to as ygp1p), a functional variant or a functional fragment thereof; the promoter of the gsy2 gene (referred to as gsy2p), a functional variant or a functional fragment thereof), molasses-regulated promoters (e.g., the promoter of the mol1gene (referred to as mol 1p), a functional variant or a functional fragment thereof), heat shock-regulated promoters (e.g., the promoter of the glo1 gene (referred to as glo1p), a functional variant or a functional fragment thereof; the promoter of the sti1 gene (referred to as sti1p), a functional variant or a functional fragment thereof; the promoter of the ygp1 gene (referred to as ygp1p), a functional variant or a functional fragment thereof; the promoter of the gsy2 gene (referred to as gsy2p), a functional variant or a functional fragment thereof), oxidative stress response promoters (e.g., the promoter of the cup1 gene (referred to as cup1p), a functional variant or a functional fragment thereof; the promoter of the ctt1 gene (referred to as ctt1p), a functional variant or a functional fragment thereof; the promoter of the trx2 gene (referred to as trx2p), a functional variant or a functional fragment thereof; the promoter of the gpd1 gene (referred to as gpd1p), a functional variant or a functional fragment thereof; the promoter of the hsp12 gene (referred to as hsp12p), a functional variant or a functional fragment thereof), osmotic stress response promoters (e.g., the promoter of the ctt1 gene (referred to as ctt1p), a functional variant or a functional fragment thereof; the promoter of the glo1 gene (referred to as glo1p), a functional variant or a functional fragment thereof; the promoter of the gpd1 gene (referred to as gpd1p), a functional variant or a functional fragment thereof; the promoter of the ygp1 gene (referred to as ygp1p), a functional variant or a functional fragment thereof), nitrogen-regulated promoters (e.g., the promoter of the ygp1 gene (referred to as ygp1p), a functional variant or a functional fragment thereof) and the promoter of the adh1 gene (referred to as adh1p), a functional variant or a functional fragment thereof.

Promoters that can be included in the heterologous nucleic acid molecule of the present disclosure include, without limitation, the promoter of the tdh1 gene (referred to as tdh1p, a functional variant or a functional fragment thereof), of the hor7 gene (referred to as hor7p, a functional variant or a functional fragment thereof), of the hsp150 gene (referred to as hsp150p, a functional variant or a functional fragment thereof), of the hxt7 gene (referred to as hxt7p, a functional variant or a functional fragment thereof), of the gpm1 gene (referred to as gpm1p, a functional variant or a functional fragment thereof), of the pgk1 gene (referred to as pgk1p, a functional variant or a functional fragment thereof), of the stl1 gene (referred to as stl1p, a functional variant or a functional fragment thereof) and/or of the tef2 gen (referred to as tef2p and having, for example, the nucleic acid sequence of SEQ ID NO: 29, a functional variant or a functional fragment thereof). In an embodiment, the promoter is or comprises the tef2p. In still another embodiment, the promoter comprises or consists essentially of the tdh1p and the hor7p. In a further embodiment, the promoter is the thd1p. In another embodiment, the promoter is the adh1p.

In the context of the present disclosure, the expression “functional fragment of a promoter” when used in combination to a promoter refers to a shorter nucleic acid sequence than the native promoter which retain the ability to control the expression of the nucleic acid sequence encoding the polypeptides during the propagation phase of the recombinant yeast host cells. Usually, functional fragments are either 5′ and/or 3′ truncation of one or more nucleic acid residue from the native promoter nucleic acid sequence.

In some embodiments, the heterologous nucleic acid molecules include one or a combination of terminator sequence(s) to end the translation of the heterologous protein (or of the chimeric protein comprising same). The terminator can be native or heterologous to the nucleic acid sequence encoding the heterologous protein or its corresponding chimera. In some embodiments, one or more terminators can be used. In some embodiments, the terminator comprises the terminator derived from is from the dit1 gene (dit1t, a functional variant or a functional fragment thereof), from the idp1 gene (idp1t, a functional variant or a functional fragment thereof), from the gpm1 gene (gpm1t, a functional variant or a functional fragment thereof), from the pma1 gene (pam1t, a functional variant or a functional fragment thereof), from the tdh3 gene (tdh3t, a functional variant or a functional fragment thereof), from the hxt2 gene (a functional variant or a functional fragment thereof), from the adh3 gene (adh3t, a functional variant or a functional fragment thereof), and/or from the ira2 gene (ira2t, a functional variant or a functional fragment thereof). In an embodiment, the terminator comprises or is derived from the dit1 gene (dit1t, a functional variant or a functional fragment thereof). In another embodiment, the terminator comprises or is derived adh3t and/or idp1t. In the context of the present disclosure, the expression “functional variant of a terminator” refers to a nucleic acid sequence that has been substituted in at least one nucleic acid position when compared to the native terminator which retain the ability to end the expression of the nucleic acid sequence coding for the heterologous protein or its corresponding chimera. In the context of the present disclosure, the expression “functional fragment of a terminator” refers to a shorter nucleic acid sequence than the native terminator which retain the ability to end the expression of the nucleic acid sequence coding for the heterologous protein or its corresponding chimera.

### Yeast Products and Compositions

The heterologous polypeptides of the present disclosure can be provided in recombinant yeasts, purified forms or in a product obtained from the recombinant yeasts. The polypeptides having thermostable alpha-amylase activity and recombinant yeast host cells comprising same can be provided in a yeast product, which can be, in some embodiments, an inactivated yeast product such as a yeast extract or an active/semi-active yeast product such as a cream yeast. In some embodiments, the yeast product is a yeast extract produced from recombinant yeast host cells expressing the polypeptides. The yeast extract may additionally include nutrients available to facilitate the growth of yeast cells. In other embodiments, the yeast product is a (substantially) purified form of the polypeptide.

As used in the context of the present disclosure, the expressions “purified form” or “isolated form” refers to the fact that the polypeptides have been physically dissociated from at least one components required for their production (such as, for example, a host cell or a host cell fragment). A purified form of the polypeptide of the present disclosure can be a cellular extract of a host cell expressing the polypeptide being enriched for the polypeptide of interest (either through positive or negative selection). The expressions “substantially purified form” or “substantially isolated” refer to the fact that the polypeptides have been physically dissociated from the majority of components required for their production (including, but not limited to, components of the recombinant yeast host cells). In an embodiment, a polypeptide in a substantially purified form is at least 90%, 95%, 96%, 97%, 98% or 99% pure.

As used in the context of the present disclosure, the expression “recombinant form” refers to the fact that the polypeptides have been produced by recombinant DNA technology using genetic engineering to express the polypeptides in the recombinant yeast host cell.

In an aspect, the polypeptides having thermostable alpha-amylase activity and recombinant yeast host cells may be provided in a composition that additionally includes a glucoamylase (either provided in a substantially purified form or expressed in a recombinant yeast host cell). A glucoamylase (EC 3.2.1.3) is an enzyme that hydrolyzes terminal 1,4-linked alpha-D-glucose residues successively from non-reducing ends of amylose chains to release free glucose. The glucoamylase may be isolated, or associated with a host cell. For example, in PCT Application No. PCT/EP2017/066378 published under WO 2018/002360, titled “ALPHA AMYLASES FOR COMBINATION WITH GLUCOAMYLASES FOR IMPROVING SACCHARIFICATION” and filed on 30 June 2017, the contents of which are herein incorporated by reference, teaches various strains of recombinant yeast host cells modified to express gluco-amylase.

When the yeast product is an inactivated yeast product, the process for making the yeast product broadly comprises two steps: a first step of providing propagated recombinant yeast host cells and a second step of lysing the propagated yeast host cells for making the yeast product. The process for making the yeast product can include an optional separating step and an optional drying step. In some embodiments, the propagated recombinant yeast host cells are propagated on molasses. Alternatively, the propagated recombinant yeast host cells are propagated on a medium comprising a yeast extract .

In some embodiments, the recombinant yeast host cells can be lysed using autolysis (which can be optionally be performed in the presence of additional exogenous enzymes). In some embodiments, the propagated recombinant yeast host cells can be lysed using autolysis. For example, the propagated recombinant yeast host cells may be subject to a combined heat and pH treatment for a specific amount of time (e.g., 24 h) in order to cause the autolysis of the propagated recombinant yeast host cells to provide the lysed recombinant yeast host cells. For example, the propagated recombinant cells can be submitted to a temperature of between about 40° C. to about 70° C. or between about 50° C. to about 60° C. The propagated recombinant cells can be submitted to a temperature of at least about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C. or 70° C. Alternatively or in combination the propagated recombinant cells can be submitted to a temperature of no more than about 70° C., 69° C., 68° C., 67° C., 66° C., 65° C., 64° C., 63° C., 62° C., 61° C. 60° C., 59° C., 58° C., 57° C., 56° C., 55° C., 54° C., 53° C., 52° C., 51° C., 50° C., 49° C., 48° C., 47° C., 46° C., 45° C., 44° C., 43° C., 42° C., 41° C. or 40° C. In another example, the propagated recombinant cells can be submitted to a pH between about 4.0 and 8.5 or between about 5.0 and 7.5. The propagated recombinant cells can be submitted to a pH of at least about, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4 or 8.5. Alternatively or in combination, the propagated recombinant cells can be submitted to a pH of no more than 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3., 5.2, 5.1, 5.0, 4.9, 4.8, 4.7, 4.6 or 4.5.

In some embodiments, the recombinant yeast host cells can be homogenized (for example using a bead-milling technique, a bead-beating or a high pressure homogenization technique) and as such the process for making the yeast product comprises an homogenizing step.

The process for making the yeast product can also include a drying step. The drying step can include, for example, with spray-drying and/or fluid-bed drying. When the yeast product is an autolysate, the process may include directly drying the lysed recombinant yeast host cells after the lysis step without performing an additional separation of the lysed mixture.

To provide additional yeast products, it may be necessary to further separate the components of the lysed recombinant yeast host cells. For example, the cellular wall components (referred to as a “insoluble fraction”) of the lysed recombinant yeast host cell may be separated from the other components (referred to as a “soluble fraction”) of the lysed recombinant yeast host cells. This separating step can be done, for example, by using centrifugation and/or filtration. The process of the present disclosure can include one or more washing step(s) to provide the cell walls or the yeast extract. The yeast extract can be made by drying the soluble fraction obtained.

In an embodiment of the process, the soluble fraction can be further separated prior to drying. For example, the components of the soluble fraction having a molecular weight of more than 10 kDa can be separated out of the soluble fraction. This separation can be achieved, for example, by using filtration (and more specifically ultrafiltration). When filtration is used to separate the components, it is possible to filter out (e.g., remove) the components having a molecular weight less than about 10 kDa and retain the components having a molecular weight of more than about 10 kDa. The components of the soluble fraction having a molecular weight of more than 10 kDa can then optionally be dried to provide a retentate as the yeast product.

When the yeast product is an active/semi-active product, it can be submitting to a concentrating step, e.g. a step of removing part of the propagation medium from the propagated recombinant yeast host cells. The concentrating step can include resuspending the concentrated and propagated recombinant yeast host cells in the propagation medium (e.g., unwashed preparation) or a fresh medium or water (e.g., washed preparation).

The yeast product can be provided as an inactive form or is created during the liquefaction/fermentation process. The yeast product can be provided in a liquid, semi-liquid or dry form.

In an aspect, the polypeptides having thermostable alpha-amylase activity and recombinant yeast host cells may be provided in a composition that additionally includes starch which can, in some embodiments, be provided in a liquefaction medium, a liquefied medium or a fermentation medium. A liquefaction medium comprises relatively intact starch molecules. A liquefied medium is a medium obtained after a liquefaction step in which the starch has been optionally heated and at least part of the starch molecules have been hydrolyzed. The viscosity of the liquefied medium is lower than the viscosity of the liquefaction medium prior to the liquefaction step. A fermentation medium comprises a liquefied medium to which a fermenting organism (such as a yeast cell) capable of metabolizing starch to produce a fermentation product (e.g., ethanol and CO₂) has been added. During the fermentation step, the starch molecules of the fermentation medium can be further hydrolyzed.

### Process for Breaking Down Starchy Material

The polypeptides and/or recombinant host cells described herein can be used to break down starch and/or dextrins into smaller molecules (e.g. by hydrolysis). The polypeptides can be used in a substantially purified form as an additive to a liquefaction process. Alternatively or in combination, the polypeptides can be expressed from one or more recombinant host cell and added to the liquefaction medium prior to the liquefaction process.

The process comprises combining a substrate to be hydrolyzed (optionally included in a liquefaction medium) with the recombinant host yeast cells expressing the polypeptides, a yeast product obtained from the recombinant yeast host cell and/or with the polypeptides in a substantially purified form. At this stage, further purified enzymes, such as, for example, non-thermostable alpha-amylases can be added also be included in the liquefaction medium.

In some embodiments, the substrate can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses or cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-protein, extensin, and pro line -rich proteins).

The substrate to be hydrolyzed comprises starch (in a gelatinized or raw form). In some embodiments, the substrate is derived from corn. In some embodiments, the use of recombinant host cells or the purified polypeptides limits or avoids the use of exogenous enzymes during fermentation to allow the breakdown of starch. The expression of the polypeptides in a recombinant host cell is advantageous because it can reduce or eliminate the need to supplement the fermentation medium with external source of purified enzymes (e.g., alpha-amylase) while allowing the fermentation of the substrate into a fermentation product (such as ethanol).

The polypeptides having thermostable alpha-amylase activity described herein can be used to increase the production of a fermentation product during fermentation. The polypeptides of the present disclosure can be used prior to, during and/or after the heating step to gelatinize the starch. The process comprises combining a substrate to be hydrolyzed (optionally included in a fermentation medium) with the polypeptide having alpha-amylase activity (either in a purified form or expressed in a recombinant host cell). In an embodiment, the substrate to be hydrolyzed is a lignocellulosic biomass. In some embodiments, the substrate comprises starch (in a gelatinized or raw form). In still another embodiment, the substrate comprises raw starch and the process includes heating (gelatinizing) the starch prior to and/or during a propagation phase of fermentation. This embodiment is advantageous because it can reduce or simplify the need to supplement the fermentation medium with external source of purified enzymes (e.g., alpha-amylase) while reducing the complexity or length of the process for fermenting the substrate into a fermentation product (such as ethanol). However, in some circumstances, it may be advisable to supplement the medium with a polypeptide having alpha-amylase activity in a purified form. Such polypeptide can be produced in a recombinant fashion in a recombinant host cell.

In some embodiments, the liquefaction of starch occurs in the presence of recombinant host cells associated with thermostable enzymes. In some embodiments, the liquefaction of starch is maintained at a temperature of between about 60° C.-105° C. to allow for proper gelatinization and hydrolysis of the crystalline starch. In an embodiment, the liquefaction occurs at a temperature of at least about 60° C., 65° C., 70° C., 75° C., 80° C., 85° C., 90° C., 95° C., 100° C. or 105° C. Alternatively or in combination, the liquefaction occurs at a temperate of no more than about 105° C., 100° C., 95° C., 90° C., 85° C., 80° C., 75° C., 70° C., 65° C. or 60° C. In yet another embodiment, the liquefaction occurs at a temperature between about 80° C. and 85° C. (which can include a thermal treatment spike at 105° C.).

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, or at least about 500 mg per hour per liter.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

The process of the present disclosure also include a process for isolating the polypeptides having alpha-amylase activity from the recombinant yeast host cell. The polypeptides obtained from such process can be used during the liquefaction step and thus introduced in the liquefaction medium. The process includes removing at least some (and in an embodiment, the majority) of the components of the recombinant yeast host cell from the heterologous polypeptides having alpha-amylase activity. Alternatively or in combination, the process includes selecting the heterologous polypeptides having alpha-amylase activity from the components of the recombinant yeast host cells. The process can include a centrifugation step, a filtration step, a washing step and/or a drying step to provide the heterologous polypeptides having alpha-amylase activity in a purified form. In embodiments in which the heterologous polypeptides having alpha-amylase activity are expressed intracellularly or associated with the recombinant yeast host cell's membrane, the process can include lysing the recombinant yeast host cells. In embodiments in which the heterologous polypeptides having alpha-amylase activity are expressed associated with the recombinant yeast host cell's membrane, the process can include disrupting the recombinant yeast host cells' membranes to purify the heterologous polypeptides having alpha-amylase activity.

### EXAMPLE I—PREPARATION AND CHARACTERISATION OF RECOMBINANT HOST CELLS AND SCREENING FOR SECRETED THERMOSTABLE ALPHA-AMYLASE ACTIVITY

Yeast cells of S. cerevisiae of strain M2390 (e.g., non-genetically modified) were modified to express a polypeptide having thermostable alpha-amylase activity. An expression cassette was prepared to incorporate a nucleic acid molecule that, when expressed, encodes a polypeptide having thermostable alpha-amylase activity. The nucleic acid molecule was incorporated into each chromosome of the M2390 cells at the FCY1 locus (e.g. 2 copies total). The native signal peptides of the polypeptides having alpha-amylase activity were replaced with the S. cerevisiae invertase signal sequence set forth in SEQ ID NO: 48. Table 1 provides a more detailed description of the yeast strains used in this example.

TABLE 1
Description of the yeast strains used in this example. All the yeast strains have been
derived from strain M2390 which is a wild-type, non-genetically modified,
Saccharomyces cerevisiae.

| | | | Copies of | | |
|---|---|---|---|---|---|
| | | | heterologous | | |
| | | | enzyme | | |
| | Heterologous | Signal | integrated | Promoter | Terminator |
| | enzyme | sequence | per | (SEQ ID | (SEQ ID |
| Name | expressed | used | chromosome | NO: 45) | NO: 47) |
| A | SEQ ID NO: 56 | SEQ ID NO: 48 | 1 | TEF2p | IDP1t |
| B | SEQ ID NO: 57 | SEQ ID NO: 48 | 1 | TEF2p | IDP1t |
| C | SEQ ID NO: 58 | SEQ ID NO: 48 | 1 | TEF2p | IDP1t |
| D | SEQ ID NO: 55 | SEQ ID NO: 48 | 1 | TEF2p | IDP1t |
| E | SEQ ID NO: 54 | SEQ ID NO: 48 | 1 | TEF2p | IDP1t |

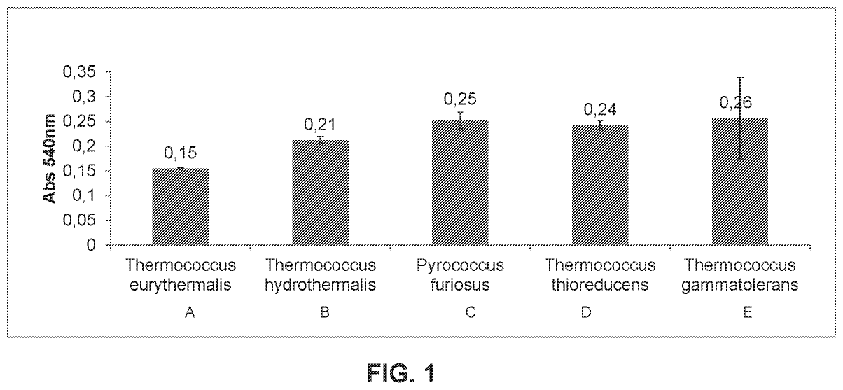

Alpha-amylase activity determination. The strains were initially grown in 600 μL of YPD₄₀ at 35° C. for 48 h in 96-well plates on a shaker at 900 rpm. To determine alpha-amylase activity, 25 μL of washed cells or cell-free supernatant was extracted from each well. In the thermo-stability treatments, the supernatants were treated for 30 mins at temperatures ranging from 79.5° C. to 99.7° C. using an Eppendorf Gradient Cycler®. Subsequently, with either the temperature-treated supernatant, the non-temperature treated supernatant or washed cells, each of these volumes was added, individually, to 100 μL of 1% raw starch with 50 mM sodium acetate buffer (pH 5.2). The reducing sugars were measured using the Dinitrosalicylic Acid Reagent Solution (DNS) method, using a 2:1 DNS:starch assay ratio and boiled at 100° C. for 5 mins. The absorbance was measured at 540 nm. As shown on FIG. 1, the supernatant of the genetically-engineered strains exhibited alpha-amylase activity at 85° C.

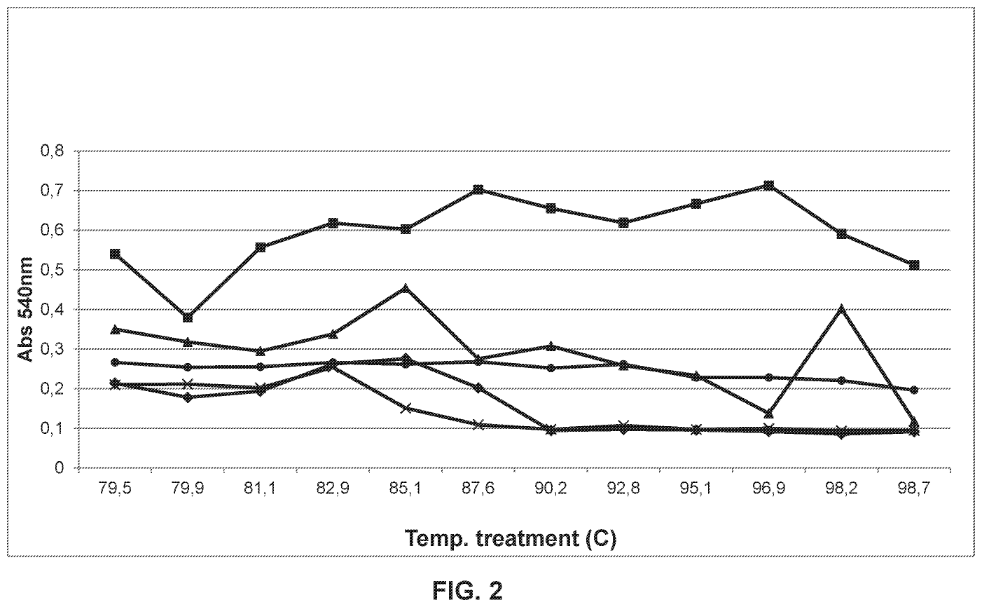

As can be seen from FIG. 2, both of the yeast-made secreted P. furiosus and T. thioreducens alpha-amylases were thermostable at temperatures of at least 99° C. The T. eurythermalis alpha-amylase lost thermostability at temperatures of above 90° C. The T. hydrothermalis alpha-amylase lost some activity at temperatures above 90° C., however retained some activity up to 99° C. The T. gammatolerans alpha-amylase began to lose thermostability at temperatures greater than 83° C. with almost complete loss in function at temperatures greater than 88° C.

### EXAMPLE II—TETHERED POLYPEPTIDES HAVING THERMOSTABLE ALPHA-AMYLASE ACTIVITY

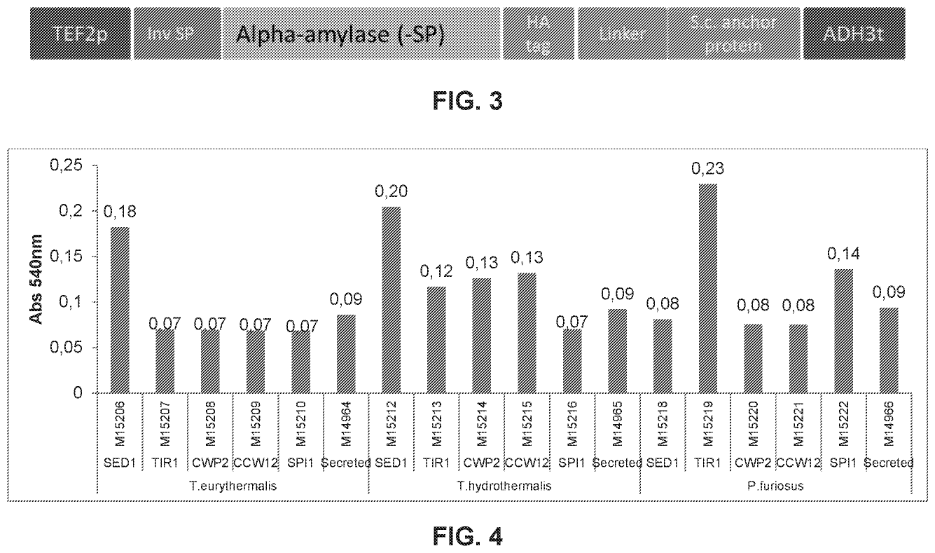

Recombinant host cells were prepared to express the polypeptides from T. eurythermalis, T. hydrothermalis and P. furiosus according to Example I, except that the polypeptides were modified to associate the polypeptide with a cell wall of the host cell using a chimeric construct as shown in FIG. 3. More specifically, the heterologous alpha-amylases lacking the signal sequence were modified to link the alpha-amylase activity portion with a GPI-anchoring portion derived from S. cerevisiae by removing the stop codon of the alpha-amylase, and fusing the alpha-amylase activity portion to SED1, SPI1, TIR1, CWP2, and CCW12 polypeptides (as set forth in SEQ ID NOs: 7, 9, 11, 13 and 15) using an HA-tag (SEQ ID NO: 49) and linker sequence (SEQ ID NO: 22) disposed there between. The expression cassette for encoding the polypeptide includes the constitutive TEF2 promoter (SEQ ID NO: 45) and ADH3 terminator (SEQ ID NO: 46).

Alpha-amylase activity was determined as indicated in Example I for the washed cells of each strain.

As seen in FIG. 4, the alpha-amylase activity of the tethered polypeptide was dependent on the particular combination of GPI-anchor and the alpha-amylase used. For the chimeric T. eurythermalis alpha-amylase, only the SED1 fusion provided cell-associated activity when compared to the secreted strain. The chimeric T. hydrothermalis alpha-amylase exhibited good cell-associated activity with the SED1, TIR1, CWP2, and CCW12 tethers. The chimeric P. furiosus alpha-amylase was active with the TIR1 and SPI1 tethers.

Recombinant host cells were prepared to express P. furiosus alpha-amylase-SPI1 chimeric polypeptide and T. hydrothermalis alpha-amylase-CCW12 chimeric polypeptides except that the GPI-anchoring portions of the tethered polypeptides were modified such that they were truncated to various lengths. Table 2 provides a more detailed description of the different strains used.

TABLE 2
Description of the strains of Example II having a truncated tethered or a modified linker. All the yeast strains have been derived from
strain M2390 which is a wild-type, non-genetically modified, Saccharomyces cerevisiae.

| | | Copies of | | | | | |
|---|---|---|---|---|---|---|---|
| | | heterologous | | | Signal | | |
| | Heterologous | enzyme | | | peptide | | |
| | enzyme | integrated per | | | (SEQ ID | | |
| Name | expressed | chromosome | Promoter | Terminator | NO: 48) | Linker | Tether |
| M15774 | Alpha-amylase | 1 | TEF2p | ADH3t | Invertase | SEQ ID | SEQ ID NO: |
| | (SEQ ID NO: 58) | | | | | NO: 22 | 30 |
| M15771 | Alpha-amylase | 1 | TEF2p | ADH3t | Invertase | SEQ ID | SEQ ID NO: |
| | (SEQ ID NO: 58) | | | | | NO: 22 | 32 |
| M15777 | Alpha-amylase | 1 | TEF2p | ADH3t | Invertase | SEQ ID | SEQ ID NO: |
| | (SEQ ID NO: 58) | | | | | NO: 22 | 34 |
| M15772 | Alpha-amylase | 1 | TEF2p | ADH3t | Invertase | SEQ ID | SEQ ID NO: |
| | (SEQ ID NO: 58) | | | | | NO: 22 | 36 |
| M15222 | Alpha-amylase | 1 | TEF2p | ADH3t | Invertase | SEQ ID | SEQ ID NO: 9 |
| | (SEQ ID NO: 58) | | | | | NO: 22 | |
| M15773 | Alpha-amylase | 1 | TEF2p | ADH3t | Invertase | SEQ ID | SEQ ID NO: |
| | (SEQ ID NO: 57) | | | | | NO: 22 | 38 |
| M15776 | Alpha-amylase | 1 | TEF2p | ADH3t | Invertase | SEQ ID | SEQ ID NO: |
| | (SEQ ID NO: 57) | | | | | NO: 22 | 40 |
| M16251 | Alpha-amylase | 1 | TEF2p | ADH3t | Invertase | SEQ ID | SEQ ID NO: |
| | (SEQ ID NO: 57) | | | | | NO: 22 | 42 |
| M15775 | Alpha-amylase | 1 | TEF2p | ADH3t | Invertase | SEQ ID | SEQ ID NO: |
| | (SEQ ID NO: 57) | | | | | NO: 22 | 44 |
| M15215 | Alpha-amylase | 1 | TEF2p | ADH3t | Invertase | SEQ ID | SEQ ID NO: |
| | (SEQ ID NO: 57) | | | | | NO: 22 | 11 |
| M15785 | Alpha-amylase | 1 | TEF2p | ADH3t | Invertase | SEQ ID | SEQ ID NO: |
| | (SEQ ID NO: 57) | | | | | NO: 22 | 11 |
| M15786 | Alpha-amylase | 1 | TEF2p | ADH3t | Invertase | SEQ ID | SEQ ID NO: |
| | (SEQ ID NO: 57) | | | | | NO: 23 | 11 |
| M15782 | Alpha-amylase | 1 | TEF2p | ADH3t | Invertase | SEQ ID | SEQ ID NO: |
| | (SEQ ID NO: 57) | | | | | NO: 24 | 11 |
| M16252 | Alpha-amylase | 1 | TEF2p | ADH3t | Invertase | SEQ ID | SEQ ID NO: |
| | (SEQ ID NO: 57) | | | | | NO: 25 | 11 |
| M16221 | Alpha-amylase | 1 | TEF2p | ADH3t | Invertase | SEQ ID | SEQ ID NO: |
| | (SEQ ID NO: 57) | | | | | NO: 26 | 11 |
| M15781 | Alpha-amylase | 1 | TEF2p | ADH3t | Invertase | SEQ ID | SEQ ID NO: |
| | (SEQ ID NO: 57) | | | | | NO: 27 | 11 |
| M16222 | Alpha-amylase | 1 | TEF2p | ADH3t | Invertase | SEQ ID | SEQ ID NO: |
| | (SEQ ID NO: 57) | | | | | NO: 28 | 11 |
| M15784 | Alpha-amylase | 1 | TEF2p | ADH3t | Invertase | SEQ ID | SEQ ID NO: 9 |
| | (SEQ ID NO: 58) | | | | | NO: 22 | |
| M15778 | Alpha-amylase | 1 | TEF2p | ADH3t | Invertase | SEQ ID | SEQ ID NO: 9 |
| | (SEQ ID NO: 58) | | | | | NO: 23 | |
| M15779 | Alpha-amylase | 1 | TEF2p | ADH3t | Invertase | SEQ ID | SEQ ID NO: 9 |
| | (SEQ ID NO: 58) | | | | | NO: 24 | |
| M15787 | Alpha-amylase | 1 | TEF2p | ADH3t | Invertase | SEQ ID | SEQ ID NO: 9 |
| | (SEQ ID NO: 58) | | | | | NO: 25 | |
| M15780 | Alpha-amylase | 1 | TEF2p | ADH3t | Invertase | SEQ ID | SEQ ID NO: 9 |
| | (SEQ ID NO: 58) | | | | | NO: 26 | |
| M15788 | Alpha-amylase | 1 | TEF2p | ADH3t | Invertase | SEQ ID | SEQ ID NO: 9 |
| | (SEQ ID NO: 58) | | | | | NO: 27 | |
| M15783 | Alpha-amylase | 1 | TEF2p | ADH3t | Invertase | SEQ ID | SEQ ID NO: 9 |
| | (SEQ ID NO: 58) | | | | | NO: 28 | |
| M15958 | Alpha-amylase | 2 | TEF2p/ | IDP1t/DIT1t | Invertase | SEQ ID | SEQ ID NO: 9 |
| | (SEQ ID NO: 58) | | ADH1p | | | NO: 22 | |
| M15206 | Alpha-amylase | 1 | TEF2p | ADH3t | Invertase | SEQ ID | SEQ ID NO: 7 |
| | (SEQ ID NO: 56) | | | | | NO: 22 | |
| M15207 | Alpha-amylase | 1 | TEF2p | ADH3t | Invertase | SEQ ID | SEQ ID NO: |
| | (SEQ ID NO: 56) | | | | | NO: 22 | 15 |
| M15208 | Alpha-amylase | 1 | TEF2p | ADH3t | Invertase | SEQ ID | SEQ ID NO: |
| | (SEQ ID NO: 56) | | | | | NO: 22 | 13 |
| M15209 | Alpha-amylase | 1 | TEF2p | ADH3t | Invertase | SEQ ID | SEQ ID NO: |
| | (SEQ ID NO: 56) | | | | | NO: 22 | 11 |
| M15210 | Alpha-amylase | 1 | TEF2p | ADH3t | Invertase | SEQ ID | SEQ ID NO: 9 |
| | (SEQ ID NO: 56) | | | | | NO: 22 | |
| M14964 | Alpha-amylase | 1 | TEF2p | ADH3t | Invertase | N/A | N/A |
| | (SEQ ID NO: 56) | | | | | | |
| M15212 | Alpha-amylase | 1 | TEF2p | ADH3t | Invertase | SEQ ID | SEQ ID NO: 7 |
| | (SEQ ID NO: 57) | | | | | NO: 22 | |
| M15213 | Alpha-amylase | 1 | TEF2p | ADH3t | Invertase | SEQ ID | SEQ ID NO: |
| | (SEQ ID NO: 57) | | | | | NO: 22 | 15 |
| M15214 | Alpha-amylase | 1 | TEF2p | ADH3t | Invertase | SEQ ID | SEQ ID NO: |
| | (SEQ ID NO: 57) | | | | | NO: 22 | 13 |
| M15216 | Alpha-amylase | 1 | TEF2p | ADH3t | Invertase | SEQ ID | SEQ ID NO: 9 |
| | (SEQ ID NO: 57) | | | | | NO: 22 | |
| M14965 | Alpha-amylase | 1 | TEF2p | ADH3t | Invertase | N/A | N/A |
| | (SEQ ID NO: 57) | | | | | | |
| M15218 | Alpha-amylase | 1 | TEF2p | ADH3t | Invertase | SEQ ID | SEQ ID NO: 7 |
| | (SEQ ID NO: 58) | | | | | NO: 22 | |
| M15219 | Alpha-amylase | 1 | TEF2p | ADH3t | Invertase | SEQ ID | SEQ ID NO: |
| | (SEQ ID NO: 58) | | | | | NO: 22 | 15 |
| M15220 | Alpha-amylase | 1 | TEF2p | ADH3t | Invertase | SEQ ID | SEQ ID NO: |
| | (SEQ ID NO: 58) | | | | | NO: 22 | 13 |
| M15221 | Alpha-amylase | 1 | TEF2p | ADH3t | Invertase | SEQ ID | SEQ ID NO: |
| | (SEQ ID NO: 58) | | | | | NO: 22 | 11 |
| M14966 | Alpha-amylase | 1 | TEF2p | ADH3t | Invertase | N/A | N/A |
| | (SEQ ID NO: 58) | | | | | | |

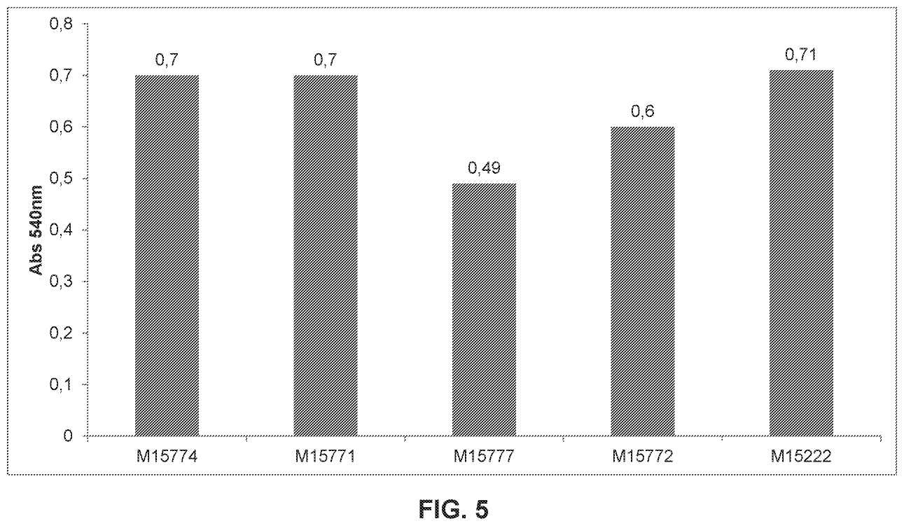

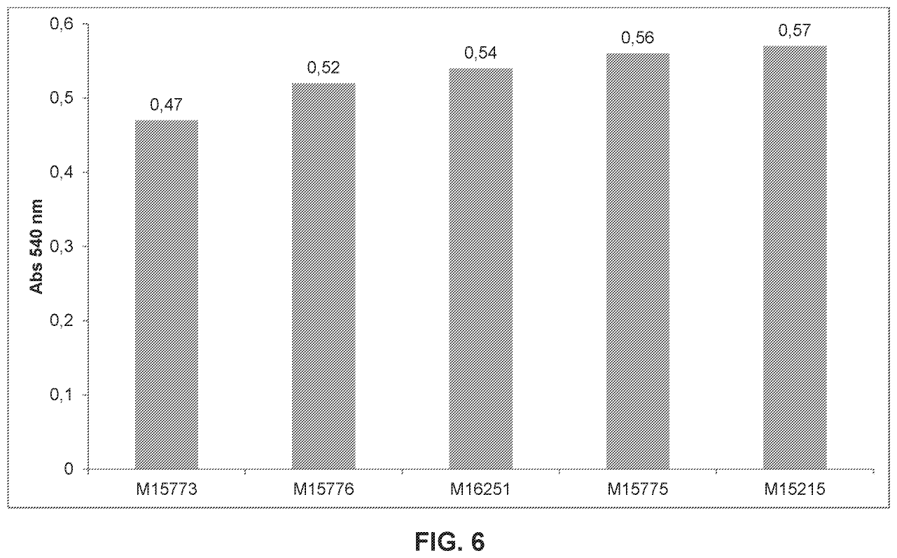

The alpha-amylase activity associated with the washed cells of the strains expressing the chimeric polypeptides with the truncated GPI anchoring portions were compared to the non-truncated GPI anchoring portion is shown in FIGS. 5 and 6.

As seen from FIG. 5, the chimeric polypeptide with the full length tethering moiety showed the same or higher alpha-amylase activity than the polypeptides with truncated tethering moieties.

As seen from FIG. 6, the chimeric polypeptides exhibited similar or higher alpha-amylase activity when compared to chimeric polypeptides having a truncated tethering moiety.

Recombinant host cells were prepared to express P. furiosus alpha-amylase-SPI1 fusion polypeptides and T. hydrothermalis alpha-amylase-CCW12 chimeric polypeptides according to Table 2, except that the chimeric polypeptide was modified to use various linkers.

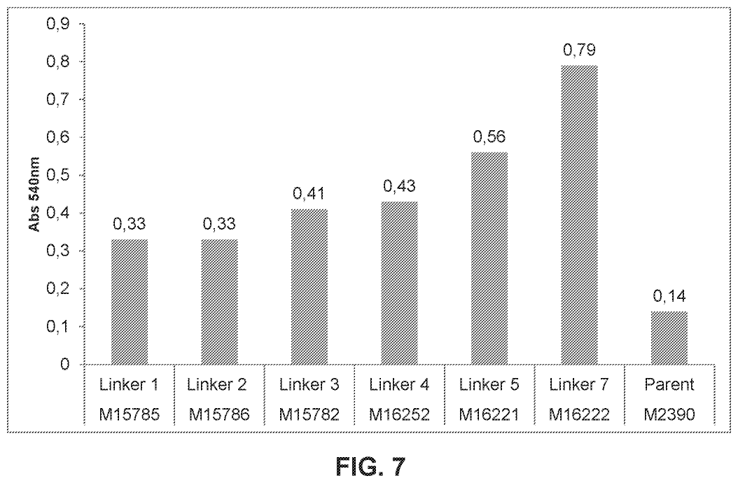

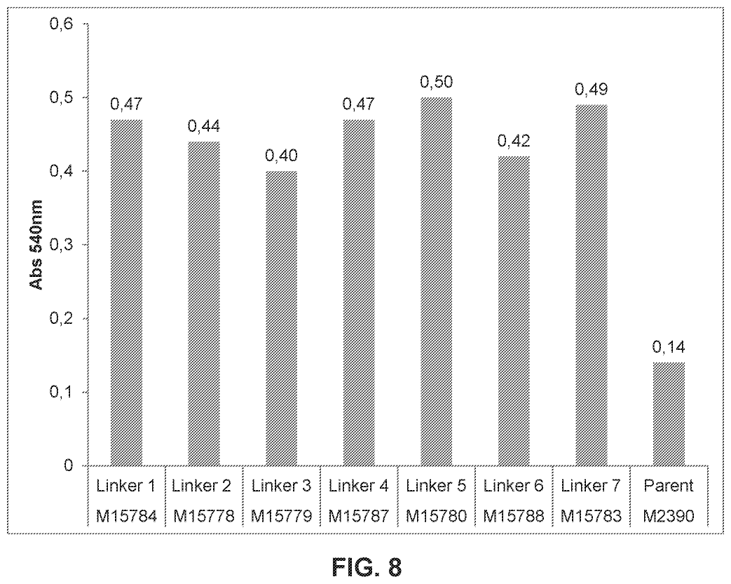

The alpha-amylase activity associated with the washed cells of the various strains expressing the chimeric polypeptides with the various linkers were compared as shown in FIGS. 7 and 8. As seen from FIG. 7, the activity was highest when the alpha-amylase activity portion was linked to the CCW12 polypeptide using the linker as set forth in SEQ ID NO: 28. As seen from FIG. 8, the activity was highest when the alpha-amylase activity portion was linked to the SPI1 polypeptide using the linker as set forth in SEQ ID NO: 26.

Lab-scale liquefaction. Cells from strain M15958 were propped in YPD overnight, centrifuged, washed, then dosed at 0.9 g dry cell weight into a 300 mL liquefaction at 85° C. Liquefactions were performed using 33% corn flour with 40% backset at pH 5.3. The slurry was raised to 60° C. and 0.9 g/L of strain M15958 added and the temperature raised 2° C/min to 85° C. Samples were run in a Dinitrosalicylic Acid Reagent Solution (DNS) assay using 25 μl of 1:8 diluted sample with 50 μl DNS and boiled for 5 mins. The absorbance was read at 540 nm and the dextrose equivalent (DE) calculated using a dextrose standard curve.

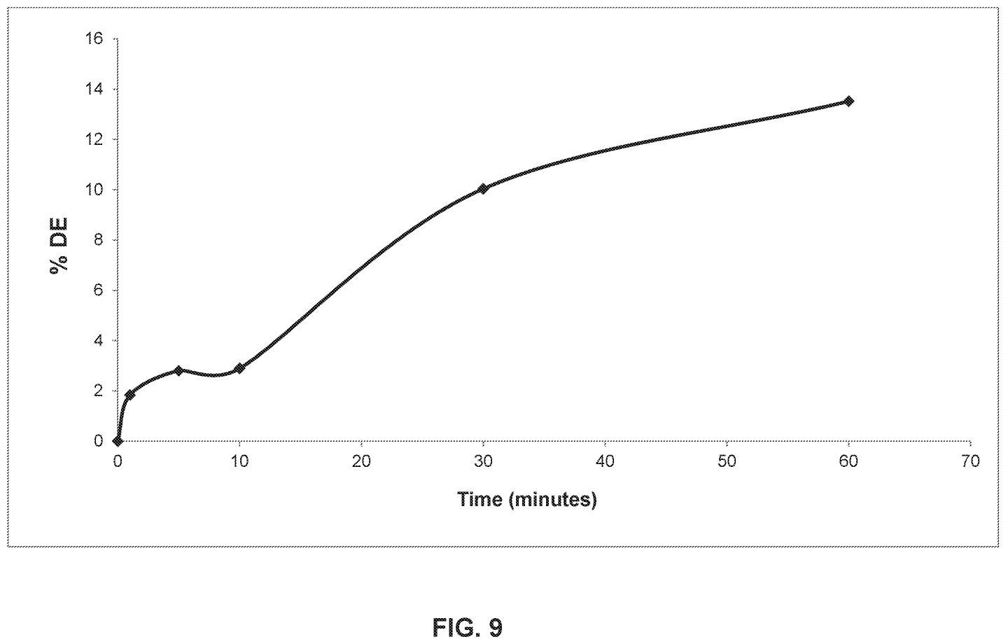

Strain M15958 was grown overnight in YPD₄₀, concentrated into a high cell density slurry with 200 g/L dry cell weight (DCW) and dosed into a lab-scale liquefaction using 0.9 g/L DCW yeast. The yeast-enzyme product was able to reach industrially relevant hydrolysis within a 60 min liquefaction without the addition of exogenous enzyme (FIG. 9).

### EXAMPLE III—SCREENING FOR SECRETED OR TETHERED THERMOSTABLE ALPHA-AMYLASE ACTIVITY IN SMALL SCALE LIQUEFACTIONS

Mini-liquefactions: Small scale liquefactions were performed using 1 g aliquots of 33% corn solids with 40% thin stillage in a 2 mL tube. Samples were taken at 1 h and evaluated for hydrolysis by measuring the reducing sugars using the DNS assay with the absorbance read at 540 nm. Strains are grown overnight in 5 mL YPD and the cells centrifuged and the pellets concentrated in spent supernatant. Cells were typically dosed at 0.03% grams of dry cell weight per grams of corn solids and the 2 mL tubes incubated at 85° C. for 1 h.

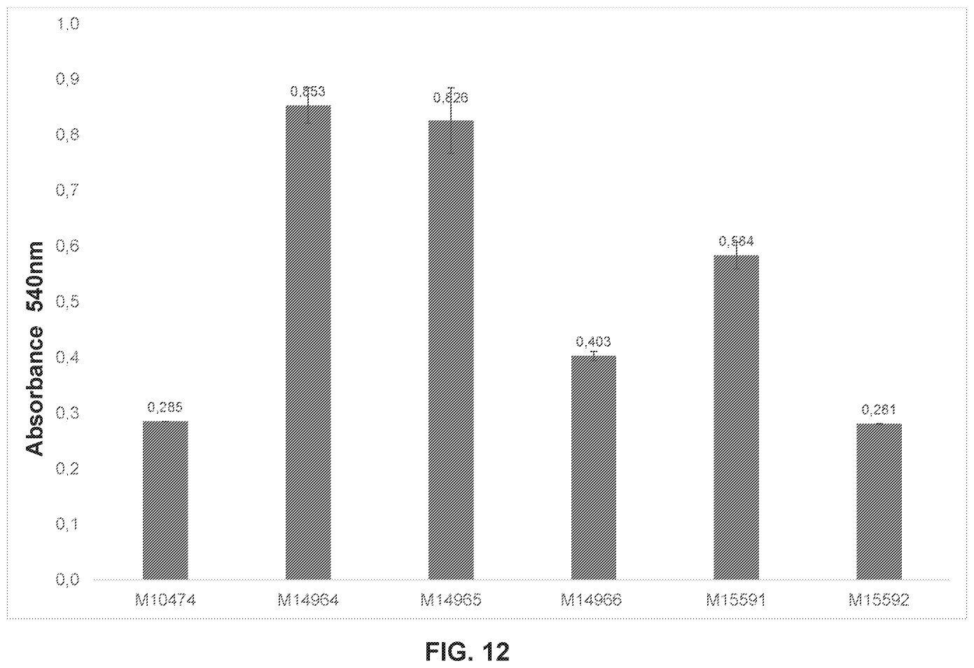

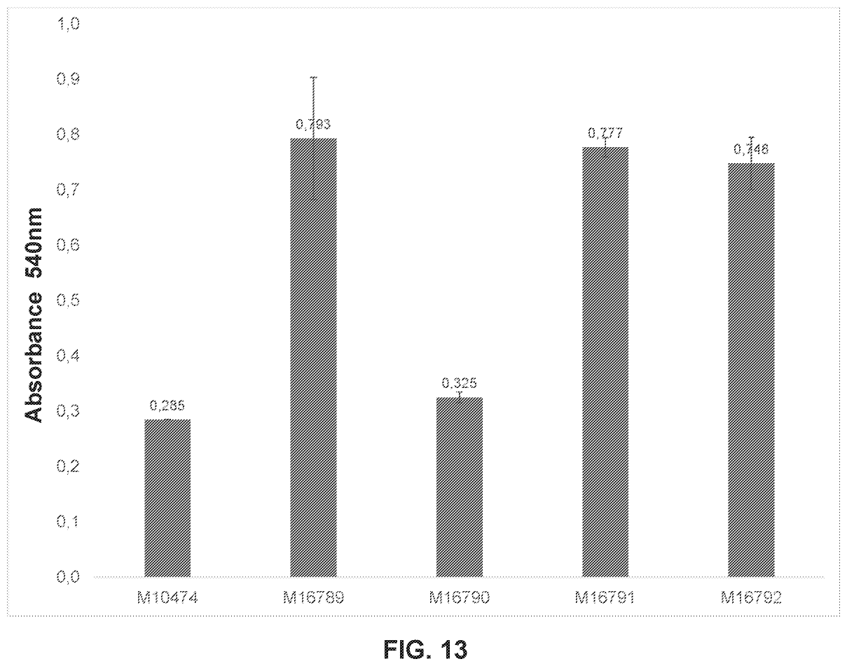

Mini-liquefactions were used to screen strains engineered with secreted thermostable alpha-amylases as well as strains engineered with tethered thermostable alpha-amylases, and compared to the parent strain, M10474. The thermostable alpha-amylases were engineered to remove their native signal sequence and replace it with the invertase signal sequence (SEQ ID NO: 41). The tethered enzymes were tethered using the AGA1 and AGA2 protein tethering complex. The strains are summarized in Table 3. Transformants were screened in a mini-liquefaction for hydrolysis on a 33% corn mash as indicated above and the results are shown in FIGS. 12 and 13.

TABLE 3
Description of the yeast strains used in this example. All the yeast strains have been
derived from strain M10474 (control) which is a wild-type, non-genetically modified,
Saccharomyces cerevisiae.

| | | Copies of | Secreted or tethered |
|---|---|---|---|
| | | heterologous | with AGA1 and AGA2 |
| | | enzyme | protein tethering |
| Strain | | integrated per | complex (SEQ ID NO: |
| Name | Heterologous enzyme origin | chromosome | 19) |
| M10474 | N/A | N/A | N/A |
| M14964 | T. eurythermalis (SEQ ID NO. 56) | 1 | Secreted |
| M14965 | T. hydrothermalis (SEQ ID NO: 57) | 1 | Secreted |
| M14966 | P. furiosus (SEQ ID NO: 58) | 1 | Secreted |
| M15591 | T. thioreducens (SEQ ID NO: 55) | 1 | Secreted |
| M15592 | T. gammatolerans (SEQ ID NO: 54) | 1 | Secreted |
| M16789 | T. hydrothermalis (SEQ ID NO: 57) | 1 | Tethered |
| M16790 | T. thioreducens (SEQ ID NO: 55) | 1 | Tethered |
| M16791 | P. furiosus (SEQ ID NO: 58) | 1 | Tethered |
| M16792 | T. gammatolerans (SEQ ID NO: 54) | 1 | Tethered |

### EXAMPLE IV—SCREENING FOR TETHERED THERMOSTABLE ALPHA-AMYLASE ACTIVITY IN LIQUEFACTIONS WITH YEAST EXTRACT

Lab-scale liquefaction: Alpha-amylase expressing yeasts were propped in YPD overnight, centrifuged, concentrated in spent supernatant, and bead beaten using 0.5 mm glass beads in an MP Biomedical benchtop homogenizer for 3 min. Bead beaten cells were dosed at 0.03% grams of dry cell weight per grams of corn solids and were added to a 300 mL liquefaction medium. Commercial thermostable alpha-amylase products (e.g., referred to as a “commercial alpha-amylase enzyme”) were used as controls with a 100% dose being 0.02% grams of enzyme per grams of total solids. Liquefactions were performed using 33% corn flour with 40% thin stillage or backset at pH 5.2 at 300 mL volumes. The slurry was raised to 70° C. followed by the commercial alpha-amylase enzyme and yeast addition, and the temperature raised 2° C/min to 85° C. where it was held for 2 h. Samples were taken after 2 h and mixed with 1% sulfuric acid to stop hydrolysis. After liquefaction, the samples were cooled to room temperature and the solids and pH adjusted to 32% and 4.8 for a subsequent fermentations.

Lab-scale fermentations: Fermentations were performed using either 25 g or 50 g of the adjusted 32% solids lab-scale liquefaction in a 100 mL serum bottle or 200 mL Pyrex® bottle, respectively. Unless stated otherwise, each fermentation typically received the same doses of 500 ppm urea, 0.6 AGU/gram total solids commercial glucoamylase, and 0.05 g/L inoculum of the M2390 strain. The fermentations were mixed at 150 rpm and incubated at 33° C. for 24 h and the temperatures dropped to 31° C. for the remainder of the fermentation. Samples were collected after 54 h and the ethanol, glycerol, and/or glucose quantified using high performance liquid chromatography (HPLC).

Dextrose equivalent measurements: Liquefactions were evaluated for hydrolysis by measuring the dextrose equivalent. Samples were evaluated for solubilized reducing sugar concentrations using the DNS assay and correlated to dextrose concentrations using a dextrose standard curve. The %DE is a measure of the amount of reducing sugars and expressed as a percentage on a dry basis relative to dextrose. The dextrose equivalent provides an indication of the average degree starch hydrolysis.

Lab-scale liquefactions were performed using strain M16449, a sister colony of M16450, expressing the tethered thermostable alpha-amylase from both P. furiosus. M16449 was constructed using a M10474 background, expressing a 2 copy per chromosome tethered P. furiosus cassette designed to express the tethered P. furiosus thermostable alpha-amylase (see Table 4). The tethered P. furiosus cassette was designed with a S. cerevisiae invertase signal peptide, HA-tag and linker sequence, along with the native SPI1 GPI anchor sequence. The two expression cassettes were designed in a parallel orientation: the first expression cassette utilized the ADH1 promoter and DIT1 terminator regulatory elements, and the second cassette utilized the TEF1 promoter and IDP1 terminator sequences. The tethered P. furiosus cassette was integrated at the FCY1 site and transformants selected YPD-5FC media.

TABLE 4
Description of the yeast extract strains used in this example. All the yeast strains have been derived from strain M10474 (control) which
is a wild-type, non-genetically modified, Saccharomyces cerevisiae.

| | | | Copies of | | | | | |
|---|---|---|---|---|---|---|---|---|
| | | | heterologous | | | | | |
| | Heterologous | | enzyme | | | | | |
| Strain | enzyme | Strain | integrated per | | | | | |
| Name | expressed | background | chromosome | Promoter | Terminator | Signal peptide | Linker | Tether |
| M10474 | N/A | N/A | N/A | N/A | N/A | N/A | N/A | N/A |
| M16449 | P. furiosus | M10474 | 2 | ADH1 | DIT1 | S. cerevisiae | SEQ ID | SPI1 |
| | alpha-amylase | | | TEF1 | IDP1 | invertase | NO: 71 | SEQ ID |
| | SEQ ID NO: 58 | | | | | SEQ ID NO: 48 | | NO: 8 |
| M19211 | P. furiosus | M16449 | 2 | ADH1 | DIT1 | S. cerevisiae | SPI1 | SEQ ID |
| | alpha-amylase | | | TEF1 | IDP1 | invertase | SEQ ID | NO: 8 |
| | SEQ ID NO: 58 | | | | | SEQ ID NO: 48 | NO: 71 | |
| | T. | | 4 | ADH1 | DIT1 | S. cerevisiae α- | SEQ ID | CCW12 |
| | hydrothermalis | | | TDH1 | IDP1 | mating factor | NO: 28 | SEQ ID |
| | alpha-amylase | | | ADH1 | DIT1 | SEQ ID NO: 70 | | NO:72 |
| | SEQ ID NO: 57 | | | TDH1 | IDP1 | | | |

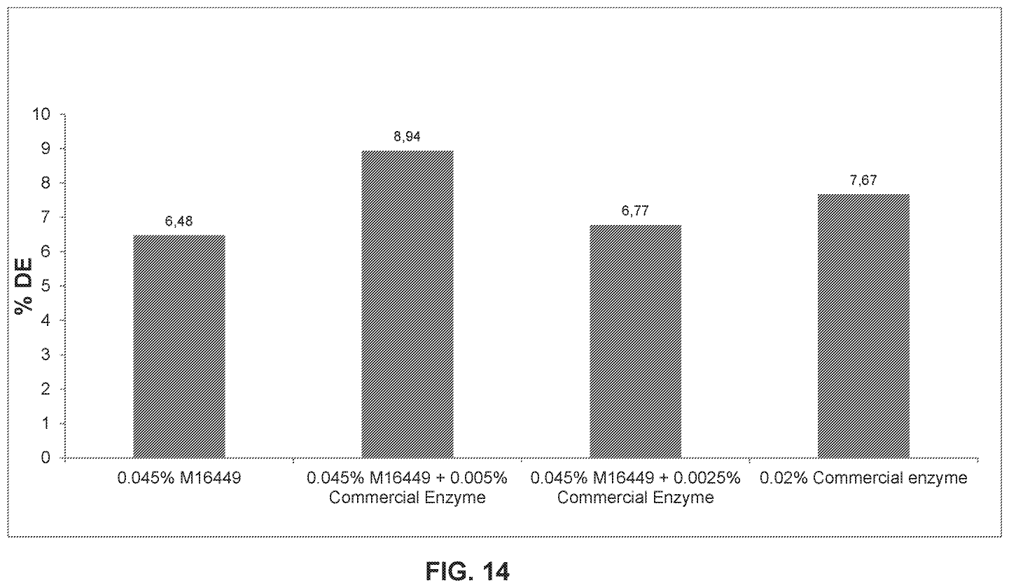

In this 300 g liquefaction, the strain used was dosed at 0.045% grams DCW/grams solids, along with two separate liquefactions supplemented with either 0.005% or 0.0025% commercial alpha-amylase enzyme and compared to the 100% (0.02% w/w) commercial dose. After 2 h at 85° C., the endpoint dextrose equivalents were measured using the DNS assay. As seen in FIG. 14, the M16449 only addition provided successful hydrolysis during liquefaction with a slightly lower DE compared to the 100% enzyme control. The addition of 0.005% commercial alpha-amylase enzyme provided the highest DE indicating more than a 75% enzyme reduction with the addition of M16449.

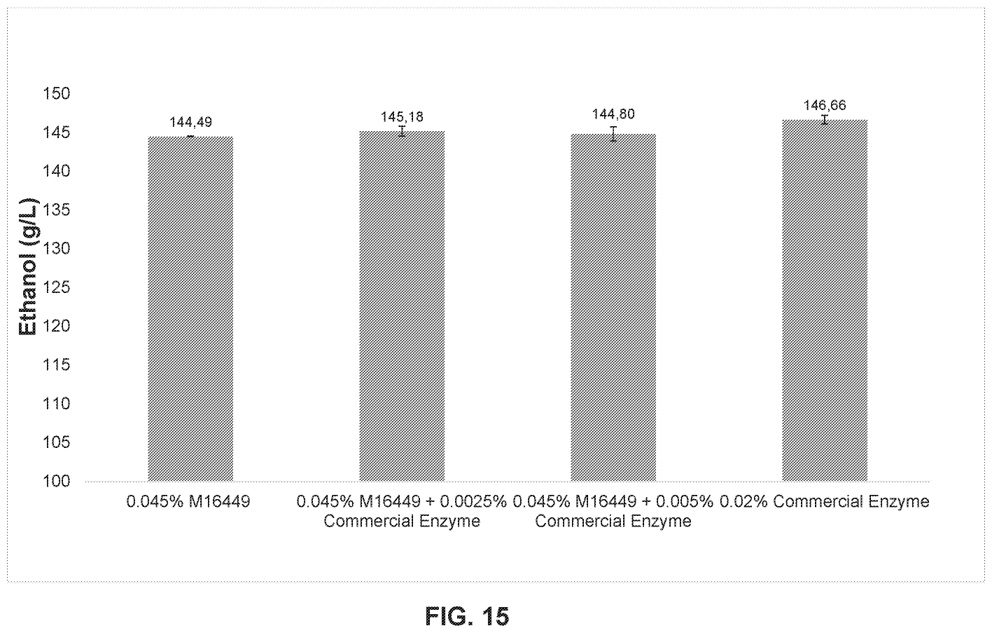

The liquefactions were subsequently fermented for 54 hours at 32% solids (TS Mash) with the addition of 500 ppm urea, pH 4.8, 33.0° C.-31° C. The M2390 strain was used in all fermentations along with a 100% glucoamylase (GA) dose. As shown in FIG. 15, the yeast hydrolyzed liquefact provided just slightly lower ethanol titers compared to the 100% commercial alpha-amylase enzyme dose indicating successful hydrolysis with a yeast only addition (FIG. 15).

A further alpha-amylase expressing strain, M19211, was engineered, co-expressing the tethered thermostable alpha-amylase from both P. furiosus and T. hydrothermalis. The M19211 strain was constructed using M16449 background expressing a 2 copy per chromosome tethered P. furiosus cassette, as indicated above, and 4 copy per chromosome T. hydrothermalis cassette designed to express the tethered T. hydrothermalis thermostable alpha-amylase (see Table 4). M16449 was premarked at the KU70 locus using a KanMX cassette with the TDK negative selection marker. The T. hydrothermalis cassette was designed with the S. cerevisiae α-mating factor signal peptide, a linker sequence, along with the native CCW12 GPI anchor sequence. The expression cassettes were designed in a convergent orientation to avoid repetitive sequences. The first expression cassette utilized the ADH1 promoter and DIT1 terminator regulatory elements. The second cassette utilized the TDH1 promoter and IDP1 terminator sequences. The third cassette also utilized the ADH1 promoter and DIT1 terminator regulatory elements in the reverse complementation. The fourth cassette utilized the TDH1 promoter and IDP1 terminator sequences in the reverse complementation. The entire T. hydrothermalis cassette was integrated at the KU70 site to remove the TDK marker and transformants selected YPD-FUDR media.

The M19211 strain was evaluated for activity in a lab-scale liquefaction. The YPD propped culture was concentrated in spent supernatant and bead beaten for 3 min using the benchtop homogenizer. The disrupted cultures were each dosed as a yeast only addition at either 0.045 or 0.03% grams DCW per grams of corn solids, along with a 0.03% DCW addition containing a 50% (0.01% weight of enzyme per weight corn solids) or 25% (0.005% w/w) dose of commercial alpha-amylase enzyme. Control liquefactions were performed using two separate commercially available alpha-amylases (commercial alpha-amylase enzyme #1 or #2), both dosed at a 100% amylase dose of 0.02% w/w commercial alpha-amylase enzyme. The changes in viscosity of the liquefaction was indirectly measured using IKA Microstar30 overhead mixers which monitor torque trends, a measurement of the power draw to maintain a set rpm, and Labworldsoft software.

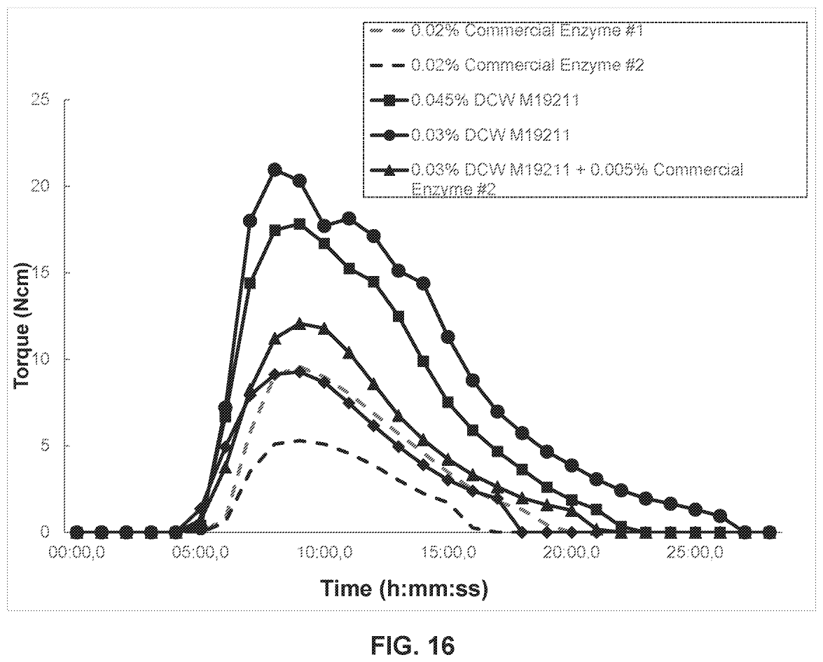

As seen in FIG. 16, the addition of the M19211 amylase-expressing yeast with either a 0.045 or 0.03% dose had a higher peak viscosity and slower viscosity break time compared to the commercial alpha-amylase enzyme doses, however, the 50 and 25% doses of commercial alpha-amylase enzyme #2 provided similar viscosity curves as the controls, demonstrating industrially relevant performance across a range of amylase products.

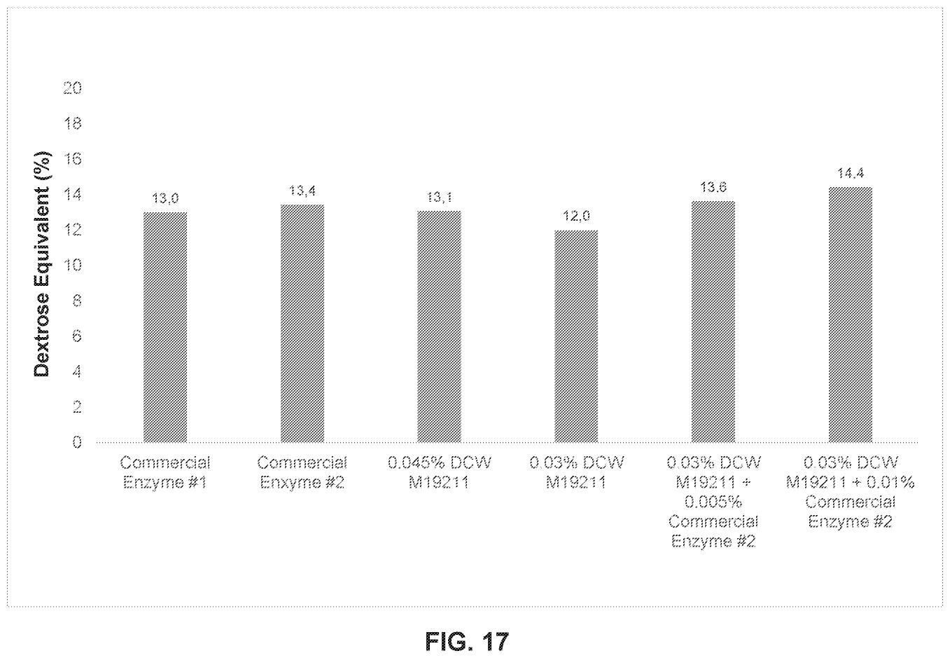

The subsequent liquefactions were evaluated for hydrolysis by measuring the dextrose equivalent. As seen in FIG. 17, each of the amylase-yeast liquefactions provided nearly equivalent or higher %DE when compared to the commercial 100% enzyme doses, indicating sufficient hydrolysis during the 2 h liquefaction.

### EXAMPLE V—COMPARISON OF DIFFERENT CELL DISRUPTION METHODS FOR INACTIVATING ALPHA-AMYLASE EXPRESSING YEAST FOR ADDITION IN LIQUEFACTIONS

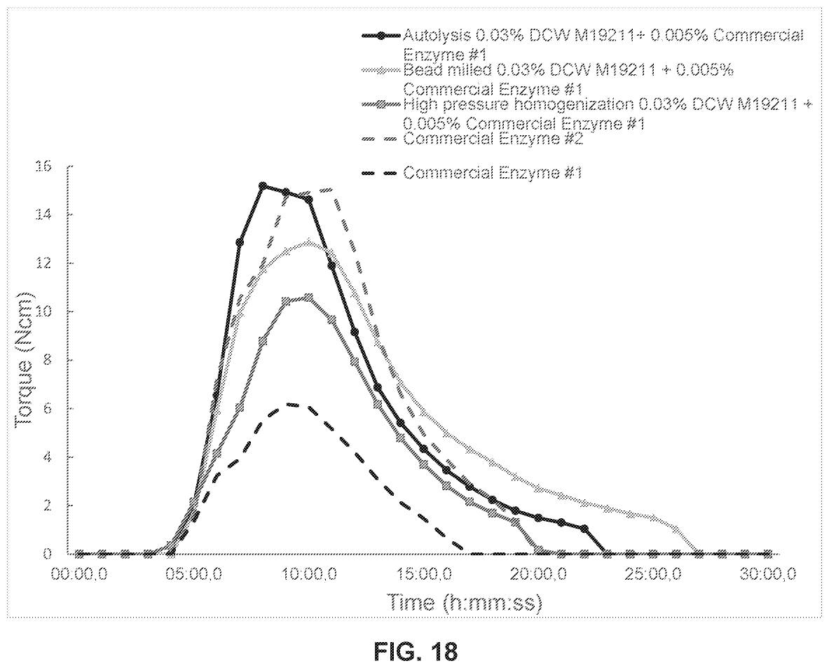

A similar lab-scale liquefaction as described previously was performed with the M19211 strain using various methods of inactivating the yeast. The yeast was prepared by either YPD propping overnight, or via a cream yeast production using molasses. The cream yeast concentrated to 20% solids in spent beer. The cream samples were disrupted using a high pressure homogenizer between 1000 and 1500 bar. The YPD propped culture were concentrated in spent supernatant and either bead beaten for 3 min using the benchtop homogenizer, or autolyzed at 70° C. for 24 h. The disrupted cultures were each dosed at 0.03% grams DCW per grams of corn solids along with a 25% dose of commercial alpha-amylase enzyme (0.005% weight of enzyme per weight corn solids). As seen in FIG. 18, the addition of the M19211 amylase-expressing yeast with a 0.005% commercial alpha-amylase enzyme provided similar viscosity curves to the full 0.02% dose of two separate commercial alpha-amylase enzymes, representing commercially relevant conditions and variations with enzyme products. The changes in viscosity is indirectly measured using IKA Microstar30 overhead mixers which monitor torque trends, which increases as the viscosity increases, and Labworldsoft software. Based on previous experiments, the 0.005% commercial alpha-amylase enzyme addition does not successfully hydrolyze the corn and maxes out the machine's torque measuring capabilities at 30Ncm and therefore was not included in this experiment. This data indicates that the disrupted M19211 cultures are capable of eliminating nearly 75% of the commercial alpha-amylase enzyme dose.

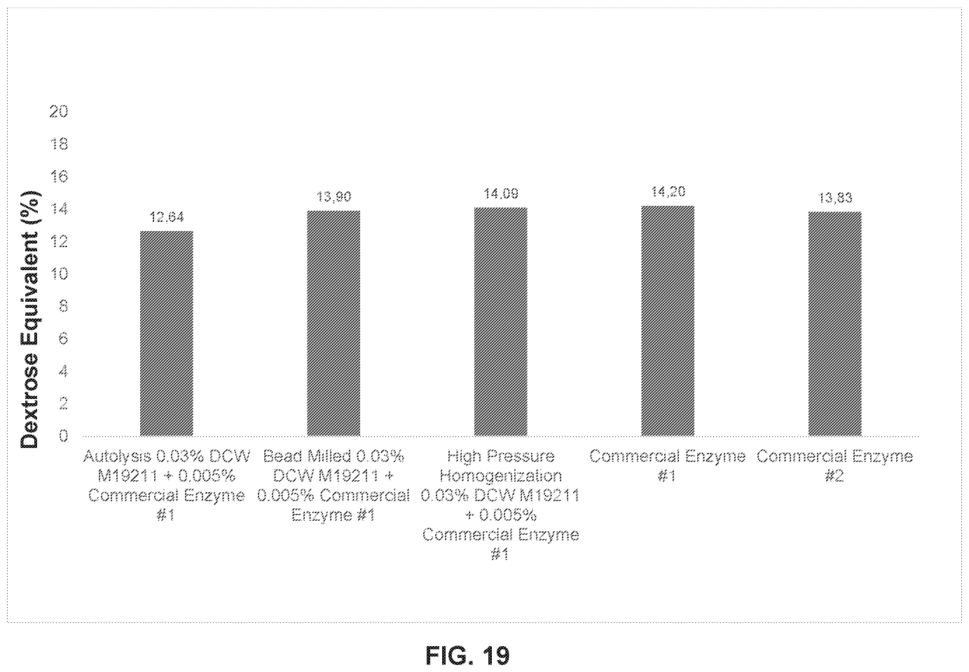

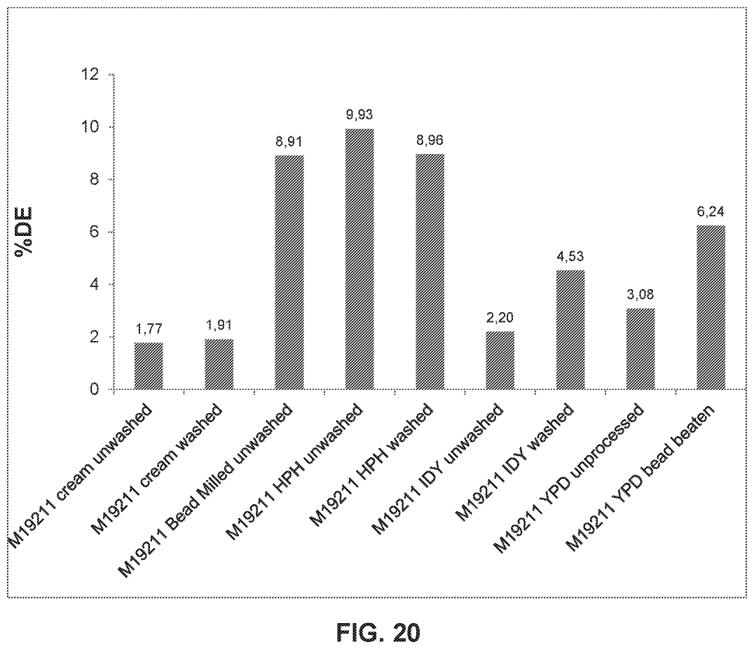

The subsequent liquefactions were evaluated for hydrolysis by measuring the dextrose equivalent. As seen in FIG. 19, each of the amylase-yeast liquefactions provided equivalent %DE when compared to the commercial 100% enzyme doses, indicating sufficient hydrolysis during the 2 h liquefaction.

M19211 was also evaluated for additional methods of processing to demonstrate potential product formats. The strain was either produced in a cream production using molasses in which the resulting cream yeast was either washed with water and resuspended to approximately 20% total DCW with water, or not washed and resuspended to 20% solids in spent beer. Both the washed and unwashed cream samples were disrupted using a high pressure homogenizer (HPH) between 1000 and 1500 bar. Both samples were also prepared into inactive dry yeast (IDY). All of these samples were compared to a YPD propped lab preparation in which the cells were either unprocessed or bead beaten for 3 mins as previously mentioned. All of the samples were compared to unprocessed cream or YPD grown cells to demonstrate an increase in activity post processing as the %DE was higher in a 1 gram mini-liquefaction (FIG. 20).

### EXAMPLE VI—YIELD IMPROVEMENTS IN FERMENTATION USING LIQUEFACTIONS CONTAINING INACTIVATED ALPHA-AMYLASE EXPRESSING YEAST

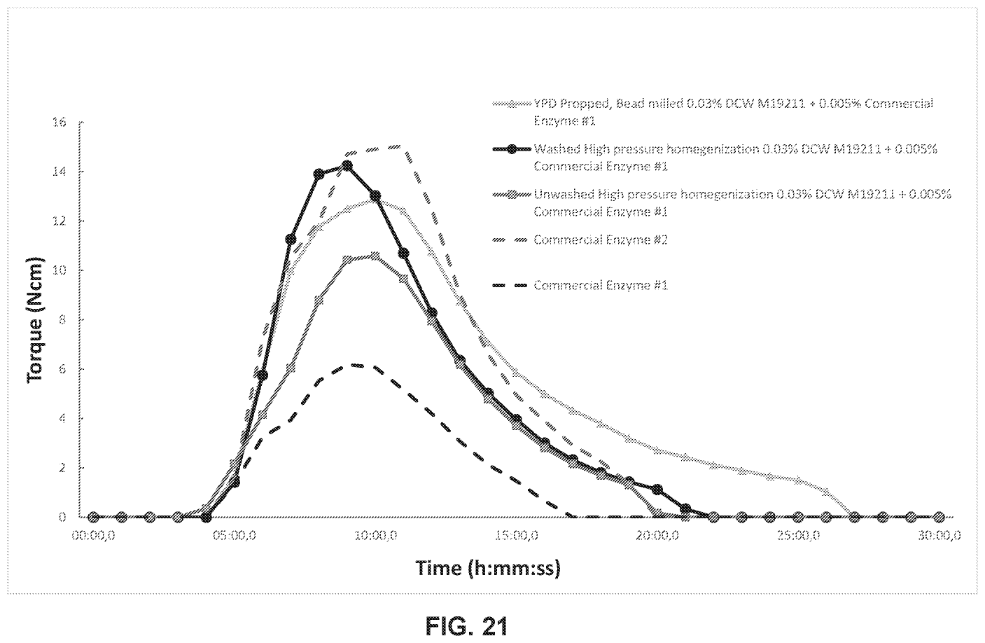

The M19211 strain, co-expressing the tethered thermostable alpha-amylase from P. furiosusand T. hydrothermalis was prepared by either YPD propping overnight, or via a cream yeast production using molasses. The cream yeast was either washed with water and resuspended to approximately 20% total DCW with water, or not washed and resuspended to 20% solids in spent beer. The cream samples were disrupted using a high pressure homogenizer between 1000 and 1500 bar. The YPD propped culture was concentrated in spent supernatant and bead beaten for 3 min using the benchtop homogenizer. The disrupted cultures were each dosed at 0.03% grams DCW per grams of corn solids along with a 25% dose of commercial alpha-amylase enzyme (0.005% weight of enzyme per weight corn solids). As seen in FIG. 21, the addition of the M19211 amylase-expressing yeast and 0.005% commercial alpha-amylase enzyme provided similar viscosity curves to the full 0.02% dose of two separate commercial alpha-amylase enzymes. The viscosity is indirectly measured using IKA Microstar30 overhead mixers which monitor torque trends, which increases as the viscosity increases. Based on previous experiments, the 0.005% commercial alpha-amylase enzyme addition does not successfully hydrolyse the corn and maxes out the machine's torque measuring capabilities at 30Ncm and therefore was not included in this experiment. This data indicates that the disrupted M19211 cultures are capable of eliminating nearly 75% of the commercial alpha-amylase enzyme dose.

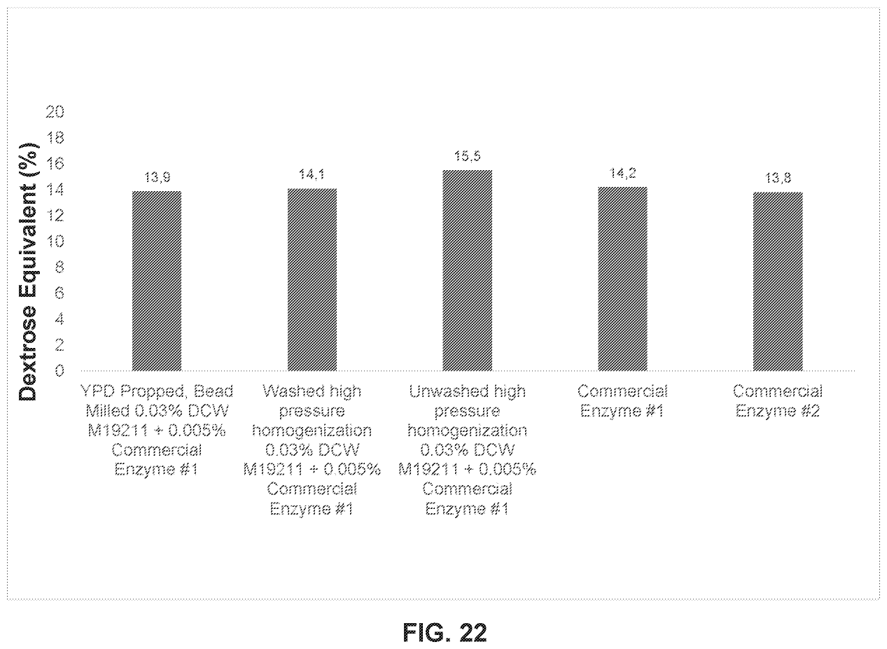

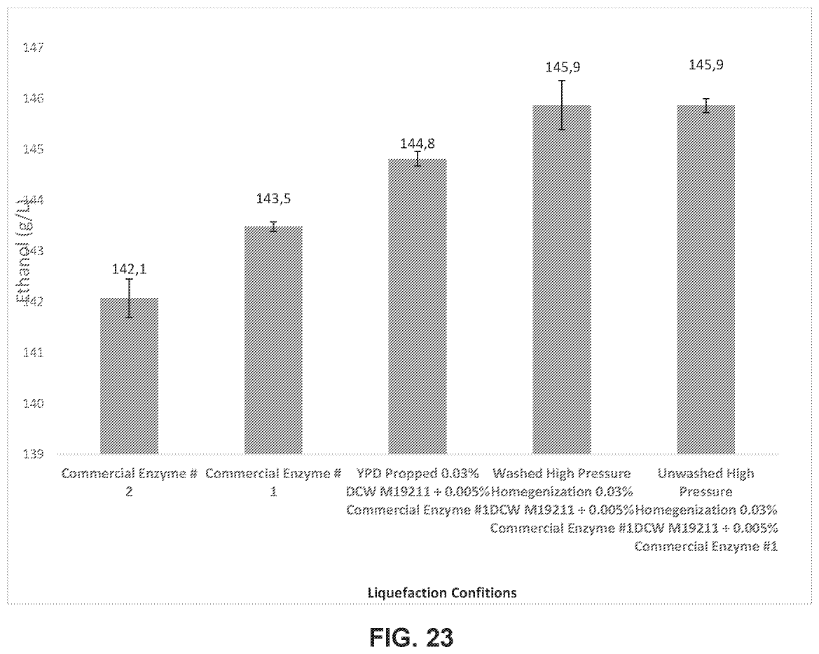

The subsequent liquefactions were evaluated for hydrolysis by measuring the dextrose equivalent. Samples were evaluated for solubilized reducing sugar concentrations using the DNS assay and correlated to glucose concentrations using a glucose standard curve. The %DE is a measure of the amount of reducing sugars and expressed as a percentage on a dry basis relative to dextrose. The dextrose equivalent gives an indication of the average degree of starch hydrolysis. As seen in FIG. 22, each of the amylase-yeast liquefactions provided equivalent or higher %DE when compared to the commercial 100% enzyme doses, indicating sufficient hydrolysis during the 2 h liquefaction. The liquefactions were subsequently fermented by adjusting the solids to 33% and fermented with the M2390 strain. The YPD-propped M19211 liquefaction provided a 1% potential ethanol yield increase relative to the 100% commercial alpha-amylase enzyme condition (Commercial alpha-amylase enzyme #1) and the disrupted M19211 cream products providing an additional 0.7% ethanol increase to the YPD propped cells, with an overall 1.7% potential ethanol increase compared to the enzyme control (FIG. 23).

### EXAMPLE VII—INTRACELLULARLY EXPRESSED POLYPEPTIDES HAVING THERMOSTABLE ALPHA-AMYLASE ACTIVITY

Intracellular expression of thermostable alpha-amylases were investigated for activity. Yeast cells of S. cerevisiae of strain M10474 were modified to express the polypeptides from P. furiosus and T. hydrothermalis, except that the native signal peptide of the P. furiosus and T. hydrothermalis alpha-amylases were replaced with a methionine to prevent secretory targeting. An expression cassette was prepared to incorporate a nucleic acid molecule that, when expressed, encodes a polypeptide having the intracellular thermostable alpha-amylases. The nucleic acid molecule was incorporated into each chromosome of the M10474 cells at the FCY1 locus (e.g. one copy per chromosome). As listed in Table 5, a series of mutations were introduced for each intracellular enzyme and subsequently engineered into the M10474 background for thermostable alpha-amylase activity analysis. Isolates were screened by growing for 72 h in YPD and evaluating whole cell cultures in a microtiter starch assay (DNS assay) at 85° C.

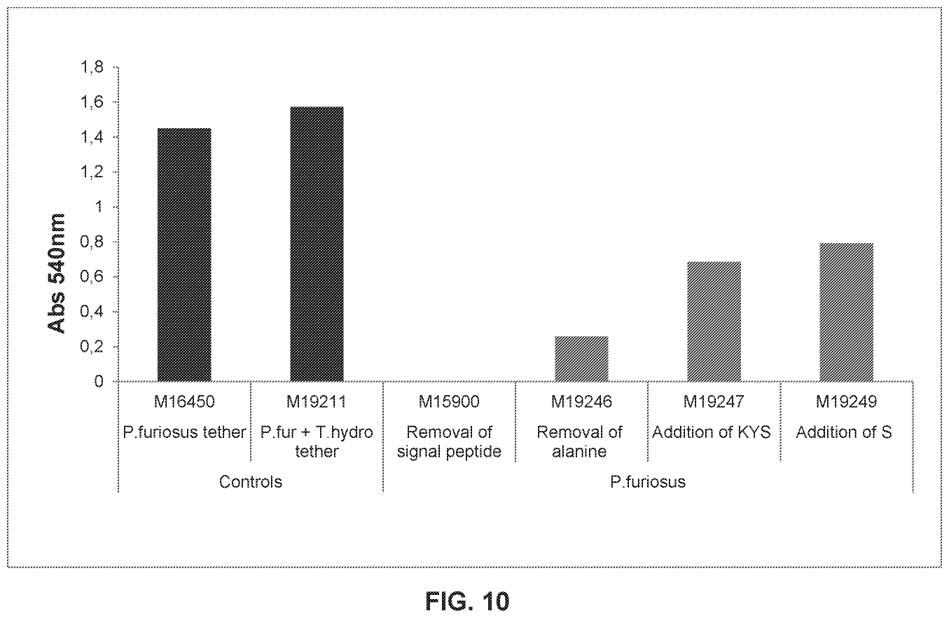

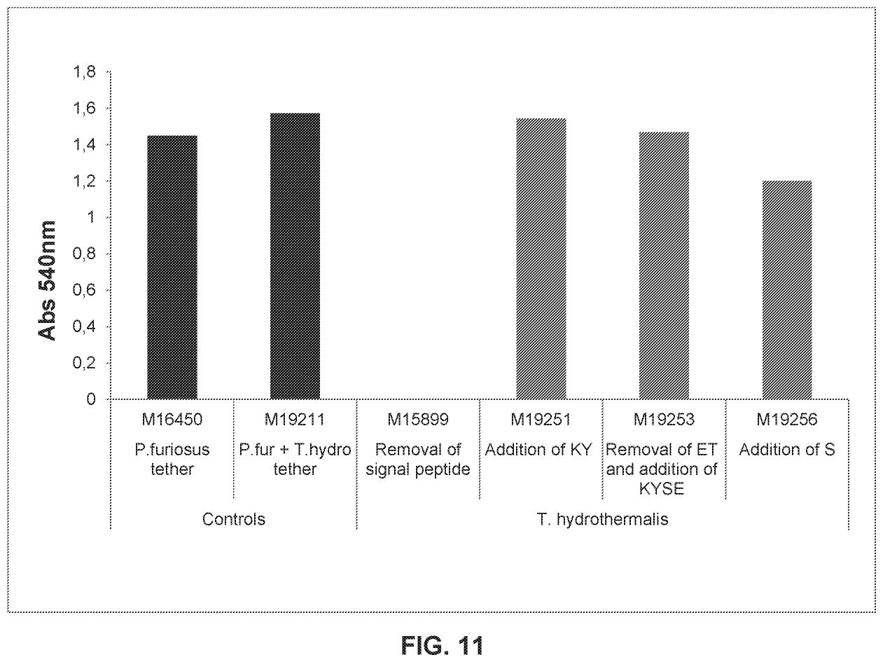

As shown in FIGS. 10 and 11, N-terminal modification for both the P. furiosus and T. hydrothermalis alpha-amylase sequences provided alpha-amylase activity to the variant polypeptides tested.

TABLE 5
Description of the yeast strains used in this example. All the yeast strains have been
originally derived from strain M10474 which is a wild-type, non-genetically modified,
Saccharomyces cerevisiae.

| | | Copies of | | |
|---|---|---|---|---|
| | | heterologous | Heterologous | |
| | Heterologous | enzyme | signal sequence | |
| Strain | enzyme | integrated per | or replaced with | N-terminal |
| Name | expressed | chromosome | Methionine (M) | modification |
| M16450 | P. furiosus alpha- | 2 | S. cerevisiae | N/A |
| | amylase | | invertase (SEQ | |
| | SEQ ID NO: 58 | | ID NO: 48) | |
| M19211 | P. furiosus alpha- | 2 | S. cerevisiae | N/A |
| | amylase | | invertase (SEQ | |
| | SEQ ID NO: 58 | | ID NO: 48) | |
| | T. hydrothermalis | 4 | S. cerevisiae α- | N/A |
| | alpha-amylase | | mating factor | |
| | SEQ ID NO: 57 | | (SEQ ID NO: 70) | |
| M15900 | P. furiosus alpha- | 1 | M | N/A |
| | amylase | | | |
| | SEQ ID NO: 63 | | | |
| M19246 | P. furiosus alpha- | 1 | M | Remove first amino acid |
| | amylase | | | residue A after the first |
| | SEQ ID NO: 64 | | | methionine. |
| M19247 | P. furiosus alpha- | 1 | M | Remove first amino acid |
| | amylase | | | residue AKYL after the |
| | SEQ ID NO: 65 | | | first methionine; addition |
| | | | | of KYS after the first |
| | | | | methionine. |
| M19249 | P. furiosus alpha- | 1 | M | Remove first amino acid |
| | amylase | | | residue A after the first |
| | SEQ ID NO: 66 | | | methionine; addition of |
| | | | | S after the first |
| | | | | methionine. |
| M15899 | T. hydrothermalis | 1 | M | N/A |
| | alpha-amylase | | | |
| | SEQ ID NO: 62 | | | |
| M19251 | T. hydrothermalis | 1 | M | Addition of KY after the |
| | alpha-amylase | | | first methionine. |
| | SEQ ID NO: 67 | | | |
| M19253 | T. hydrothermalis | 1 | M | Remove first two amino |
| | alpha-amylase | | | acid residue ET after |
| | SEQ ID NO: 68 | | | the first methionine; |
| | | | | addition of KYSE after |
| | | | | the first methionine. |
| M19256 | T. hydrothermalis | 1 | M | Addition of S after the |
| | alpha-amylase | | | first methionine. |
| | SEQ ID NO: 69 | | | |

Any documents referenced herein are incorporated by reference in their entirety. To the extent that teachings in any references incorporated by reference are in conflict with the teachings of the present disclosure, the references are incorporated insofar as they do not conflict present disclosure and the teachings of the present disclosure shall govern.

While the invention has been described in connection with specific embodiments thereof, it will be understood that the scope of the claims should not be limited by the preferred embodiments set forth in the examples, but should be given the broadest interpretation consistent with the description as a whole.

1. A recombinant yeast host cell comprising a heterologous nucleic acid molecule encoding a heterologous polypeptide having thermostable alpha-amylase activity, wherein the heterologous polypeptide comprises the amino acid sequence of:
SEQ ID NO: 57, a variant or a fragment thereof;
SEQ ID NO: 58, a variant or a fragment thereof;
SEQ ID NO: 54, a variant or a fragment thereof;
SEQ ID NO: 55, a variant or a fragment thereof; or
SEQ ID NO: 56, a variant or a fragment thereof.

2. (canceled)

3. The recombinant yeast host cell of claim 1 expressing a first heterologous polypeptide and a second heterologous polypeptide.

4. The recombinant yeast host cell of claim 3, wherein the first heterologous polypeptide has the amino acid sequence of SEQ ID NO 57, is a variant or a fragment thereof and the second heterologous polypeptide has the amino acid sequence of SEQ ID NO: 58, is a variant thereof or a fragment thereof.

5. The recombinant yeast host cell of claim 1, wherein the heterologous polypeptide further comprises a signal sequence.

6. The recombinant yeast host cell of claim 5, wherein the signal sequence is a native signal sequence.

7. The recombinant yeast host cell of claim 6, wherein the heterologous polypeptide comprises the amino acid sequence of:
SEQ ID NO: 4, a variant or a fragment thereof;
SEQ ID NO: 5, a variant or a fragment thereof;
SEQ ID NO: 1, a variant or a fragment thereof;
SEQ ID NO: 2, a variant or a fragment thereof; or
SEQ ID NO: 3, a variant or a fragment thereof.

8. The recombinant yeast host cell of claim 5, wherein the signal sequence is an heterologous signal sequence.

9. The recombinant yeast host cell of claim 8, wherein the heterologous signal sequence is from an invertase protein, an AGA2 protein, or an alpha-mating factor protein.

10. The recombinant yeast host cell of claim 9, wherein the heterologous signal sequence has the amino acid sequence of SEQ ID NO: 48, 51, or 70, a variant thereof, or a fragment thereof.

11. The recombinant yeast host cell of claim 1, wherein the heterologous polypeptide is a secreted polypeptide or a cell-associated polypeptide.

12. (canceled)

13. The recombinant yeast host cell of claim 11, wherein the cell-associated polypeptide is a membrane-associated polypeptide.

14. The recombinant yeast host cell of claim 13, wherein the membrane-associated polypeptide is a tethered heterologous polypeptide.

15.-17. (canceled)

18. The recombinant yeast host cell of claim 14, wherein the tethered heterologous polypeptide comprises a tethering moiety from a SED1 protein, a SPI1 protein, a CCW12 protein, a CWP2 protein, a TIR1 protein, a PST1 protein, a combination of a AGA1 and a AGA2 protein, or a variant thereof or a fragment thereof.

19.-21. (canceled)

22. The recombinant yeast host cell of claim 1, wherein the heterologous polypeptide is an intracellular polypeptide.

23. The recombinant yeast host cell of claim 22, wherein the heterologous polypeptide has the amino acid sequence of any one of SEQ ID NO: 59 to 63 and wherein:
the first amino acid residue after the methionine residue at position 1 has been removed;
the first two consecutive amino acid residues after the methionine residue at position 1 have been removed; and/or
at least one lysine, tyrosine, serine, or glutamic acid residue has been added after the methionine residue at position 1.

24. The recombinant yeast host cell of claim 23, wherein the heterologous polypeptide has the amino acid sequence of:
SEQ ID NO: 64, a variant or a fragment thereof;
SEQ ID NO: 65, a variant or a fragment thereof;
SEQ ID NO: 66, a variant or a fragment thereof;
SEQ ID NO: 67, a variant or a fragment thereof;
SEQ ID NO: 68, a variant or a fragment thereof; or
SEQ ID NO: 69, a variant or a fragment thereof.

25. The recombinant yeast host cell of claim 1, wherein the heterologous polypeptide has alpha-amylase activity at a temperature of at least 60° C., 65° C., 70° C., 75° C., 80° C., 85° C., 90° C., 95° C. or 99° C.

26. The recombinant yeast host cell of claim 1, wherein the heterologous nucleic acid molecule is operatively associated with an heterologous promoter sequence for allowing the expression of the polypeptide during propagation.

27. The recombinant yeast host cell of claim 1, wherein the heterologous nucleic acid molecule further encodes a chimeric protein comprising the heterologous polypeptide fused to a starch binding domain.

28. (canceled)

29. The recombinant yeast host cell of claim 1, which is a cell of genus Saccharomyces sp. or a cell of species Saccharomyces cerevisiae.

30.-32. (canceled)

33. A composition comprising the recombinant yeast host cell of claim 1 and at least one of a glucoamylase or starch.

34. A yeast product made from the recombinant yeast host cell of claim 1.

35.-36. (canceled)

37. A process for hydrolyzing starch, the process comprising contacting the recombinant yeast host cell of claim 1 with a medium comprising starch.

38.-44. (canceled)
